SEQUENCE LISTING (24 sequences)

SEQ ID NO: 1 - DNA, 1575 bp, Zea mays
  CDS (1)..(1575)
atgggcatggctgctccgcgcgcgccgctgcctctcccccaactgctgctcctcctcgttgccgcgctcctcgccgccgctcgcctccctagggcggccagggcggacgagttcgatgtccgccgccacctctccaccgtcaccaggtatgatgtggccagggagtccagtagtgtcatctccatgccgtcaatcccagacgggtgccgtgtcattcacctcaatttagtggcaagacatgggactcgcgctcctaccaagaagcgcatcaaggagctggatagattggcagttcgactggaagcccttctgaaagaggcgaatcaggtccttgatagtgattctctgaagaaaattccatcctggattaaaggctgggaatcacgctggaagggtaggactaaaggtggtgagctgattagtgaaggggaagaggagctttacaatttagctaccagaatgagggagaggtttcaagatctatttgatgacgaatatcaccctgatgtatattcaataagagcaacccaggttcctcgagcatcagctagtgcagtggcatttgggttgggactactttctgggaaaggaaagcttggacaagggaagaaccgagccttttctgttctgagtgagagtcgtgcaagtgatatttgtctgagattctttgacagctgtgagacatacaaggcatacaggaaaaggaaggagcctgatgtagagaagcaaaaggaaccaattctagagcatgtcacagctgcacttgtcaatcgttatcacctaaaatttacaactcgcgatgtttcttccctctggtttctttgtaagcaggaaacatctttgttgaatacaacaaatcaagcttgtgggctttttaatgaagctgaggttcgttttctggagtggacagatgatttggagggttttgttctaaaaggctatggtgagtcaattaactacaggatgggactgccattgctcaaggatgttgtccagtcaatggaagaagcaatcatagctagagaagaaaaccgtgctgatggtacgtttgaaaaggcaaggctccgatttgcacatgcagaaactgttgttccttttagctgccttcttggtctttttcttgaaggtccagaaattgagaagatacagagagaggaagcattggacctaccccctttgccgccacagggaagaaactggaagggcagtgttgttgcgcctttcgctggtaacaatatgctggttttatatcaatgtccaagcaaaatttcggatggcagcacaatctctggaggccgaaacaactcttacttagttcaagttctacacaacgaagtcccagtttcaatgcctgggtgcggcaacaaagatttctgtccgttcgaggagttcaaggagaaaattgtgaaaccgcacctgaagcacgactacaacatgatatgcaaggtcaaatccccagcggcaagcgaggagcctgcctcgttcgcctccagggtgtccagtttcttcctaggactcctctcgcagaaagggtaccgcggtgtgggcgccgagggcgtcaagaccgagctgtag

SEQ ID NO: 2 - protein, 524 aa, Zea mays
MGMAAPRAPLPLPQLLLLLVAALLAAARLPRAARADEFDVRRHLSTVTRYDVARESSSVISMPSIPDGCRVIHLNLVARHGTRAPTKKRIKELDRLAVRLEALLKEANQVLDSDSLKKIPSWIKGWESRWKGRTKGGELISEGEEELYNLATRMRERFQDLFDDEYHPDVYSIRATQVPRASASAVAFGLGLLSGKGKLGQGKNRAFSVLSESRASDICLRFFDSCETYKAYRKRKEPDVEKQKEPILEHVTAALVNRYHLKFTTRDVSSLWFLCKQETSLLNTTNQACGLFNEAEVRFLEWTDDLEGFVLKGYGESINYRMGLPLLKDVVQSMEEAIIAREENRADGTFEKARLRFAHAETVVPFSCLLGLFLEGPEIEKIQREEALDLPPLPPQGRNWKGSVVAPFAGNNMLVLYQCPSKISDGSTISGGRNNSYLVQVLHNEVPVSMPGCGNKDFCPFEEFKEKIVKPHLKHDYNMICKVKSPAASEEPASFASRVSSFFLGLLSQKGYRGVGAEGVKTEL

SEQ ID NO: 3 - DNA, 1509 bp, Oryza sativa
  CDS (1)..(1509)
atggctgctccccgcacgcctctccccctcgtcctcctcctcgtctccgccgcgttcgacgtccgccgccacctctccaccgtcaccaggtacgatgtggcgaggggatccaatagtgtgtcctccgcgccgtctatgtcggatgagtgccgcgtgatccacctcaatctcgtggcaagacatgggactcgcgcacctaccaaaaagagaatcaaagagctggatagactggcggttcggttgaaggctcttatcgatgaagcaaaacaagggcctgaaagtgactccctgaaaaaaattccttcatggatgaaagggtgggagtcaccctggaaaggtagggtgaaaggtggtgagctggtcagtgaaggggaggaagagctatacaaccttgctatcagagtcaaggagaggtttcaaggcctatttgatgaggaatatcaccctgatgtgtattcaataagagcaactcaggttcctcgggcatcagctagtgcagtagcatttggtttgggtctactttctgggaaagggaagcttggacctgtgaaaaaccgtgccttttctgttctgagtgagagtcgtgcaagtgatatttgtctgcgattctttgatagctgtgaaacatacaaggactacaggaaaagaaaggagcctgatgttgaaaagcaaaaggaaccaattttagaacacgtcacatcggcattagttaaccgttatcatctcaattttacaccaaaagatgtttcttccctctggttcctttgcaagcaggaagcatctttaatgaatataaccaatcaagcttgtcaactttttaatgaagctgaggtttattttctagagtggacagatgatctggagggctttgtgctaaaaggttatggtgagtcaataaactatcggatgggactgccattgctcaaggacgttgtccagtcaatggaagaagcaatcgttgctaaagaagaaaaccaccctgatggtacatatgagaaggcaaggctccgatttgcacatgctgaaactgttgtccctttctcatgtcttcttggtctttttcttgaaggatcagattttgcgaagatacaacgggaggaatcattggacatacctcctgtgccaccacagggaagaaattggaagggcagtgttgttgcaccttttgctggtaacaatatgttggctttgtaccagtgcccaggaaaaactgatggtggtaagatttctcgggatcagaagagctcatacttcgtgcaggttatacacaatgaagctccagtttcaatgccgggatgcgggaacaaagatttctgcccatttgaagagttcaaggagaagatagttgaaccccacctgaagcatgactacgacgccctatgcaagataaggccggtggcaagagaggagccttcctccttcagttccaggatgtccaatttcttcctaggtttgttctcgcagaaaggataccgtgttagtgctcaggatgtgaagtcggagctgtag

SEQ ID NO: 4 - protein, 502 aa, Oryza sativa
MAAPRTPLPLVLLLVSAAFDVRRHLSTVTRYDVARGSNSVSSAPSMSDECRVIHLNLVARHGTRAPTKKRIKELDRLAVRLKALIDEAKQGPESDSLKKIPSWMKGWESPWKGRVKGGELVSEGEEELYNLAIRVKERFQGLFDEEYHPDVYSIRATQVPRASASAVAFGLGLLSGKGKLGPVKNRAFSVLSESRASDICLRFFDSCETYKDYRKRKEPDVEKQKEPILEHVTSALVNRYHLNFTPKDVSSLWFLCKQEASLMNITNQACQLFNEAEVYFLEWTDDLEGFVLKGYGESINYRMGLPLLKDVVQSMEEAIVAKEENHPDGTYEKARLRFAHAETVVPFSCLLGLFLEGSDFAKIQREESLDIPPVPPQGRNWKGSVVAPFAGNNMLALYQCPGKTDGGKISRDQKSSYFVQVIHNEAPVSMPGCGNKDFCPFEEFKEKIVEPHLKHDYDALCKIRPVAREEPSSFSSRMSNFFLGLFSQKGYRVSAQDVKSEL

SEQ ID NO: 5 - DNA, 22 bp, Artificial Sequence
  Description of Artificial Sequence Synthetic oligonucleotide
catatgggcatggctgctccgc

SEQ ID NO: 6 - DNA, 26 bp, Artificial Sequence
  Description of Artificial Sequence Synthetic oligonucleotide
ggatcctaggtcttgacgcctacagc

SEQ ID NO: 7 - DNA, 19 bp, Artificial Sequence
  Description of Artificial Sequence Synthetic oligonucleotide
caagacatgggactcgcgc

SEQ ID NO: 8 - DNA, 19 bp, Artificial Sequence
  Description of Artificial Sequence Synthetic oligonucleotide
cgcgagtcccatgtcttgc

SEQ ID NO: 9 - DNA, 22 bp, Artificial Sequence
  Description of Artificial Sequence Synthetic oligonucleotide
caaatcgattcgccatggctgc

SEQ ID NO: 10 - DNA, 27 bp, Artificial Sequence
  Description of Artificial Sequence Synthetic oligonucleotide
ggatcctacagctccgacttcacatcc

SEQ ID NO: 11 - DNA, 21 bp, Artificial Sequence
  Description of Artificial Sequence Synthetic oligonucleotide
catatggctgctccccgcacg

SEQ ID NO: 12 - DNA, 29 bp, Artificial Sequence
  Description of Artificial Sequence Synthetic oligonucleotide
ggatcctacagctccgtcttaacatcttg

SEQ ID NO: 13 - DNA, 25 bp, Artificial Sequence
  Description of Artificial Sequence Synthetic oligonucleotide
gtctatcgtttctattaagccagac

SEQ ID NO: 14 - DNA, 478 bp, Artificial Sequence
  Description of Artificial Sequence Synthetic EST 3′TaMIPP sequence
ggcacgagccgggggtggcagcagaggaggagccatcctctttcagctccaagctcaacttcttccttgatctgctctcgcggaaaggttaccgttttaaggggcaagatgttaagacggagctgtagagtacaggcgccttgtgccgcgacgaccttggattaggactgacatgtggacactaaccttggtgtttttgtacctaggtttggtgacttgtgagcgagttcagcgcgtatcaggctctgatggcttgcaggtgccgccgtgtgcgagtctggcttaatagaaacgatagactactcatattaataaggaattcttttttcggaaaaaaaaaaaaaaaaaaactcgagagtacttntagagcggccgcgggcccatcgattttncacccgggtggggtaccaggtaagtgtacccaattcgccctatagtgagtcgnattacaattcactggcccgcgntttacaacgtn

SEQ ID NO: 15 - protein, 25 aa, Artificial Sequence
  Description of Artificial Sequence Signal peptide
MGMAAPRAPLPLPQLLLLLVAALLA

SEQ ID NO: 16 - protein, 18 aa, Artificial Sequence
  Description of Artificial Sequence Signal peptide
MAAPRTPLPLVLLLVSAA

SEQ ID NO: 17 - DNA, 1575 bp, Zea mays
  CDS (1)..(1575)
atgggcatgactgcgccgcgcgcgccgctgcctctcccccaactgctgctcctcctcgttgccgcgctcctcgccgccgctcccctccctagggcggccagggcggacgagttcgacgtccgccgccacctctccaccgtcaccaggtatgatgtggccagggagtccagtagtgtcatctccatgccgtcaatcccagacgggtgccgtgtcattcacctcaatttagtggcaagacatgggactcgcgctcctaccaagaagcgcatcaaggagctggatagattggcagttcgactggaagcccttctgaaagaggcaaatcaggtccttgatagtgattctctgaagaaaattccatcctggattaaaggctgggaatcacgctggaagggtaggactaaaggtggtgagctgattagtgaaggggaagaggagctttacaatttagctaccagaatgagggagaggtttcaagatctatttgatgacgaatatcaccctgatgtatattcaataagagcaacccaggttcctcgagcatcagctagtgcagtggcatttgggttgggactactttctgggaaaggaaagcttggacaagggaagaaccgagccttttctgttctgagtgagagtcgtgcaagtgatatttgtctgagattctttgacagctgtgagacatacaaggcatacaggagaaggaaggagcctgatgtagagaagcaaaaggaaccaattctagagcatgtcacagctgcacttgtcaatcgttatcacctaaaatttacaactcgcgatgtttcttccctctggtttctttgtaagcaggaagcatctttgttgaatacaacaaatcaagcttgtgggctttttaatgaagctgaggttcgttttctggagtggacagatgatttggagggttttgttctaaaaggctatggtgagtcaattaactacaggatgggactgccattgctcaaggatgttgtccagtcaatggaagaagcaatcatagctagagaagaaaaccgtgctgatggtacgtttgaaaaggcaaggctccgatttgcacttgcagaaactgttgttccttttagctgccttcttggtctttttcttgaaggtccagaaattgagaggatacagagagaggaagcattggacctaccccctttgccgccacagggaagaaactggaagggcagtgttgttgcgccttttgctggtaacaatatgctggttttatatcaatgtccaagcaaaatttcggatggcagcacaatctctggaggccgaaacaactcttacttagttcaagttctacacaacgaagtcccagtttcaatgcctgggtgcggcaacaaagatttctgtccgttcgaggagttcaaggagaaaattgtgaaaccgcacctgaagcacgactacaacatgatatgcaaggtcaaatccccagcggcaagcgaggagcctgcctcgttcgcctccagggtgtccagtttcttcctaggactcctctcgcagaaagggtaccgcggtgtgggcgccgagggcgtcaagaccgagctgtag

SEQ ID NO: 18 - protein, 524 aa, Zea mays
MGMTAPRAPLPLPQLLLLLVAALLAAAPLPRAARADEFDVRRHLSTVTRYDVARESSSVISMPSIPDGCRVIHLNLVARHGTRAPTKKRIKELDRLAVRLEALLKEANQVLDSDSLKKIPSWIKGWESRWKGRTKGGELISEGEEELYNLATRMRERFQDLFDDEYHPDVYSIRATQVPRASASAVAFGLGLLSGKGKLGQGKNRAFSVLSESRASDICLRFFDSCETYKAYRRRKEPDVEKQKEPILEHVTAALVNRYHLKFTTRDVSSLWFLCKQEASLLNTTNQACGLFNEAEVRFLEWTDDLEGFVLKGYGESINYRMGLPLLKDVVQSMEEAIIAREENRADGTFEKARLRFALAETVVPFSCLLGLFLEGPEIERIQREEALDLPPLPPQGRNWKGSVVAPFAGNNMLVLYQCPSKISDGSTISGGRNNSYLVQVLHNEVPVSMPGCGNKDFCPFEEFKEKIVKPHLKHDYNMICKVKSPAASEEPASFASRVSSFFLGLLSQKGYRGVGAEGVKTEL

SEQ ID NO: 19 - protein, 4 aa, Artificial Sequence
  Description of Artificial Sequence Illustrative peptide
KDEL

SEQ ID NO: 20 - protein, 4 aa, Artificial Sequence
  Description of Artificial Sequence Illustrative peptide
KTEL

SEQ ID NO: 21 - protein, 6 aa, Artificial Sequence
  Description of Artificial Sequence Illustrative peptide
SEKDEL

SEQ ID NO: 22 - protein, 6 aa, Artificial Sequence
  Description of Artificial Sequence Illustrative peptide
HEKDEL

SEQ ID NO: 23 - protein, 7 aa, Artificial Sequence
  Description of Artificial Sequence Consensus sequence
RHGXRXP

SEQ ID NO: 24 - protein, 4 aa, Artificial Sequence
  Description of Artificial Sequence Illustrative peptide
KSEL

(19) United States
(12) Patent Application Publication
(10) Pub. No.: US 2004/0132153 A1
(43) Pub. Date: Jul. 8, 2004

(54) Nucleic acids coding for a plant phosphatase of the mipp type with phytase activity and uses

(76) Inventors: Pascual Perez, Chanonat (FR); Tania Di Gioia, Massy (FR)

Correspondence Address: YOUNG & THOMPSON, 745 SOUTH 23RD STREET 2ND FLOOR, ARLINGTON, VA, 22202

(21) Appl. No.: 10/312,731

(22) PCT Filed: Jul. 2, 2001
(86) PCT No.: PCT/FR01/02116

(30) Foreign Application Priority Data
Jun. 30, 2000 (FR) 00/08529

(51) Int. Cl. C12N 9/16; C07H 21/04; C12N 5/04

(52) U.S. Cl. 435/196; 435/69.1; 435/320.1; 536/23.2; 435/419

(57) ABSTRACT

The invention concerns nucleic acid sequences coding for a MIPP type plant phosphatase with phytase activity, the isolated proteins encoded by said sequences, and their uses, in particular for improving food digestibility in animals.

The present invention relates to nucleic acid sequences encoding a plant phosphatase of the MIPP type with phytase activity, to the isolated proteins encoded by these sequences, and also to the uses thereof.

This invention relates more specifically to nucleic acid sequences from maize, rice and wheat, encoding a MIPP.

The importance of the role of phosphorus in animal nutrition has been known for a long time. Phosphorus is essential to animal growth; 80% of the phosphorus is located in the skeleton. The remainder of the phosphorus is contained in the soft tissues, where it is involved in many biochemical reactions, such as the synthesis of nucleic acids, phospholipids and certain B vitamins.

Animals obtain phosphorus from food, mainly from plants. Most of the phosphate present in plants, in particular in seeds, is in the form of phytin, a complex salt of myoinositol hexakisphosphoric acid or phytic acid. Phytin therefore constitutes a store of phosphorus and of sugars, but also of various cations (Ca²⁺, Zn²⁺, Mg²⁺, Fe³⁺)

Phytin, a salt of phytic acid complexed with various cations, therefore represents the major storage form of phosphate in the seeds, but it is also present in pollen, storage organs such as tubers or in roots. Its distribution in seeds varies from one species to the other:

in dicotyledons (soybean, castor oil plant), it is mainly present in the cotyledons and the albumen;

in cereals (wheat, rice), it is present especially in the aleurone, whereas it is absent from the albumen;

in maize, the majority of the phytin present in the grain is stored in the embryo (O'Dell-et al., 1972; Barba et al., 1997).

In maize grains, the phosphorus of phytate represents up to 88% of the total phosphorus (O'Dell et al., 1972). Phytin degradation is carried out by specific phosphatases, including phytases, which are enzymes capable of hydrolyzing the phosphate from phytic acid (Gibson and Ullah, 1990), releasing myoinositol and inorganic phosphate.

However, phosphatases of the plant phytase type are produced in insufficient amounts in the parts of plants used to feed certain animals.

In particular, the phytin and phytic acid which come from dry seed flours are not digested by monogastric animals (such as pigs and chickens) in the absence of exogenous phosphatase, and are therefore excreted unmodified. They thus contribute to the pollution of soil and water by phosphate, in areas where intensive rearing takes place. In addition, phytic acid is considered to be an antinutritional factor since it chelates mineral elements and causes protein aggregation. Consequently, these minerals and proteins will not be correctly assimilated, during intestinal transit, by monogastric animals (Graf, 1986).

Recent studies have been undertaken in order to allow better digestibility of phytin and of phytic acid in monogastric animals. Many studies have related mainly to phosphatases or other types of enzyme involved in phytin mobilization. For example, genes encoding cereal phytases have been isolated. Identification of the Phyt I and Phyt II genes, originating from maize, have thus made it possible to obtain maize plants having an increased phytase content (WO 98/05785).

As for the authors of the present invention, they have focused their attention on another family of enzymes, which do not exhibit any significant homology with Phyt I or Phyt II: MIPPs—Multiple Inositol Polyphosphate Phosphatases, related to histidine phosphatases recorded only in the animal kingdom. They play an important role in ossification processes, in particular in the development and differentiation of chondrocytes (Romano et al., 1998). These phosphatases are also known to be the only animal enzymes which hydrolyze molecules of inositol tetra-, penta- and hexaphosphate (Chi et al., 1999).

Although these enzymes were, until now, acknowledged to be specific for the animal species, the authors of the present invention have succeeded in characterizing plant nucleic acid sequences exhibiting significant homology with the genes encoding animal MIPPs. Thus, the enzymes encoded by these plant nucleic acids will subsequently be referred to as plant MIPPs.

Phytin, a preferred substrate for MIPPs, thus releases inorganic phosphate and also the chelated cations. The inorganic phosphate and the various cations released are then available for the metabolic pathways which require them.

A subject of the present invention is therefore an isolated nucleic acid encoding a plant MIPP enzyme with phytase activity. MIPP enzymes from cereals are particularly targeted, in particular those from maize, from rice and from wheat. A subject of the invention is more particularly an isolated nucleic acid comprising a sequence selected from SEQ ID No. 1 (maize cDNA), SEQ ID No. 3 (rice cDNA) or SEQ ID No. 17 (maize cDNA).

Preferentially, a subject of the invention is an isolated nucleic acid comprising the sequence SEQ ID No. 17 or a sequence homologous to the sequence SEQ ID No. 17.

The sequences complementary to these nucleic acid sequences are also part of the present invention.

A subject of the invention is also fragments of the above sequences encoding peptides which conserve the activity mentioned, or the sequences complementary to these fragments.

The invention also relates to the sequences homologous to the sequences mentioned above.

The term “homologous” refers to any nucleic acid having one or more sequence modification(s) with respect to all or part of a given sequence.

These homologous sequences are preferentially defined as:

i) sequences similar to at least 70% of the sequence SEQ ID No. 1, No. 3 or No. 17, preferably at least 80%, even more preferably at least 90%; or

ii) sequences which hybridize with the sequence SEQ ID No. 1, No. 3 or No. 17, or the sequence complementary thereto, under stringent hybridization conditions, or

iii) sequences encoding a plant MIPP enzyme comprising the amino acid sequence SEQ ID No. 2, No. 4 or No. 18, or a homologous amino acid sequence.

Preferentially, such a homologous nucleotide sequence hybridizes specifically to the sequences complementary to the sequence dSEQ ID No. 1, No. 3 or No. 17, under stringent conditions. The parameters which define stringency conditions depend on the temperature at which 50% of the paired strands separate (Tm).

For sequences comprising more than 30 bases, Tm is defined by the equation:

Tm=81.5+0.41(% G+C)+16.6Log(cation concentration)−0.63(% formamide)−(600/number of bases) (Sambrook et al., 1989).

For sequences less than 30 bases long, Tm is defined by the equation: Tm=4(G+C)+2 (A+T).

Under suitable stringency conditions, at which a specific sequences do not hybridize, the hybridization temperature may preferably be from 5 to 10° C. below Tm, and the hybridization buffers used are preferably solutions of high ionic strength, such as a 6×SSC solution for example.

The term “sequences similar” used above refers to the complete resemblance, or identity, between the nucleotides compared, but also to the incomplete resemblance which is described as similarity. This search for similarity in the nucleic acid sequences distinguishes, for example, purines and pyrimidines.

A nucleotide sequence homologous to the ORF represented in SEQ ID No. 1, No. 3 or No. 17 therefore includes any nucleotide sequence which differs from sequence SEQ ID No. 1, No. 3 or No. 17 by mutation, insertion, deletion or substitution of one or more bases, or by degeneracy of the genetic code, as long as it encodes a polypeptide having the phytase activity of the plant MIPP.

Included among such homologous sequences are the sequences of the genes of cereals other than maize or rice, and also the allelic variants.

A subject of the present invention is also an isolated polypeptide, named plant MIPP, preferably comprising the amino acid sequence SEQ ID No. 2, No. 4 or No. 18.

More particularly, the polypeptide comprises the amino acid sequence SEQ ID No. 18. It is understood that also included are the homologous sequences advantageously defined as

i) the sequences similar to at least 70% of the sequences SEQ ID No. 2, No. 4 or No. 18, preferably at least 80%, even more preferably at least 90%, or

ii) the sequences encoded by a homologous nucleic acid sequence as defined above, i.e. a nucleic acid sequence which hybridizes with sequence No. 1, No. 3 or No. 17 or the sequence complementary thereto, under stringent hybridization conditions.

Here again, the term “similar” refers to the complete resemblance, or identity, between the amino acids compared, but also to the incomplete resemblance which is described as similarity. This search for similarities in a polypeptide sequence takes into account conservative substitutions, which are substitutions of amino acids of the same class, such as substitutions of amino acids with uncharged side chains (such as asparagine, glutamine, serine, threonine or tyrosine), of amino acids with basic side chains (such as lysine, arginine or histidine), of amino acids with acidic side chains (such as aspartic acid or glutamic acid), or of amino acids with apolar side chains (such as glycine, alanine, valine, leucine, isoleucine, proline, phenylalanine, methionine, tryptophan or cysteine).

More generally, the expression “homologous amino acid sequence” is therefore intended to mean any amino acid sequence which differs from the sequence SEQ ID No. 2, No. 4 or No. 18 by substitution, deletion and/or insertion of an amino acid or of a small number of amino acids, in particular by substitution of natural amino acids with unnatural amino acids or pseudo amino acids at positions such that these modifications do not significantly harm the biological activity of the plant MIPP.

Homology is generally determined using a sequence analysis program (for example, Sequence Analysis Software Package of the Genetics Computer Group, University of Wisconsin Biotechnology Center, 1710 University Avenue, Madison, Wis. 53705). Similar amino acid sequences are aligned so as to obtain the maximum degree of homology (i.e. identity or similarity, as defined above). For this purpose, it may be necessary to artificially introduce gaps into the sequence. Once the optical alignment has been carried out, the degree of homology is established by recording all the positions for which the amino acids of the two sequences compared are identical, relative to the total number of positions.

The expression “the biological activity of the plant MIPP” refers in particular to its phytase activity, which can be determined by assaying the phosphate released by the enzyme from sodium phytate.

The polypeptide of the present invention may be synthesized by all the methods well known to those skilled in the art. The polypeptide of the invention may, for example, be synthesized by synthetic chemistry techniques, such as Merrifield-type synthesis, which is advantageous for reasons of purity and lack of undesired by-products and for its ease of production.

A recombinant protein may also be produced using a method in which a vector containing a nucleic acid according to the invention, such as a nucleic acid comprising the sequence SEQ ID No. 1, No. 3 or No. 17 or a homologous sequence, is transferred into a prokaryotic or eukaryotic host cell, which is cultured under conditions which allow expression of the corresponding polypeptide. The protein produced can then be recovered and purified. The purification methods used are known to those skilled in the art. The recombinant polypeptide obtained can be purified from cell lysates and extracts or from the culture medium supernatant, by methods used individually or in combination, such as fractionation, chromatography methods, etc.

A subject of the present invention is therefore also a method for producing a plant MIPP protein, characterized in that it comprises the steps of:

a) transforming a cell, in particular of a plant or of a microorganism, with an expression cassette comprising regulatory sequences capable of controlling the expression of an increased amount of MIPP in said host cell;

b) optionally culturing said host cell or, when this host cell is a plant cell, growing the transformed plant;

c) extracting the MIPP protein from the cell culture or from the transformed plant.

A subject of the present invention is therefore also an expression cassette, characterized in that it comprises at least one of the nucleic acid sequences or fragments as defined above, placed under the control of at least one regulatory sequence capable of controlling the expression of the plant MIPP protein.

Among these regulatory sequences, mention may be made of promoters, activators and introns, and transcription terminators.

A large number of promoters can be used to transform the plants according to the present invention. By way of example, mention may be made of:

promoters for constitutive expression:

p35S promoter (Kay et al., 1987)

pUbi1 promoter of the gene encoding maize ubiquitine 1 (Christensen et al., 1992)

pAct1 promoter of the gene encoding rice actin 1 (McElroy et al., 1991)

histone promoters (EP 0 507 698)

CsVMC promoter of the cassaya vein mosaic virus (Verdaguer et al., 1996, 1998)

promoters specific for an organ or for a tissue of the plant, and more particularly grain-specific promoters (Datla et al., 1997), for instance the promoters of genes encoding storage proteins such as: a wheat or barley HMWG (high molecular weight glutenin) specific for albumen (Roberts et al., 1989; Anderson O. D. et al., 1989), napin, phaseolin, helianthinin, albumin, oleosin, GEA1 and GEA6 of Arabidopsis thaliana (Gaubier et al., 1993), maize γ-zein, the pHyPRP promoter of the gene encoding a maize hybrid proline rich protein (HyPRP: Hybrid Proline Rich Protein) which is specific for the embryo and more particularly for the scutellum (José-Estanyol et al., 1992), and the Vp1 promoter of the viviparous-1 gene (transcription activator—McCarty D. R. et al. (1989)) combined with the first intron Sh of the Shrunken gene (Maas et al. (1991)), which is specific for the aleurone.

promoters which are inducible:

during a water stress (Kasuga et al., 1999)

by light: promoter of the gene encoding the RUBISCO, ribulose 1,5-BISphosphate carboxylase oxygenase, small subunit, promoter of the gene encoding chlorophyll a/b,

promoters of the genes encoding Agrobacterium tumefaciens mannopine synthase, nopaline synthase, octopine synthase,

promoters of genes encoding enzymes preferably selected from those below: maize alcohol dehydrogenase 1 (Adh1), phenylalanine ammonia lyase (PAL), HMG-CoA reductase (HMG), chitinase, glucanase, protease inhibitors, genes of the PR1 family, the gene vspB (U.S. Pat. No. 5,670,349), HMG2 (U.S. Pat. No. 5,670,349), apple beta-galactosidase (Abgl), or aminocyclopropane carboxylate synthase (WO 98/45445).

Thus, tissue-specific or organ-specific ectopic expression allows the production of seeds rich in inorganic phosphate or enriched with MIPP protein, capable of degrading phytin while at the same time limiting the possible physiological damage due to a variation in the phytin content.

In general, use will preferably be made of a constitutive promoter such as the pAct 1 promoter of the rice Act 1 gene contained in the plasmid pAct1-F4 (Mc Elroy et al., 1991) or the p35S promoter (Kay et al., 1987), or a tissue-specific promoter such as the wheat or barley HMWG promoter, or else the PCRU promoter of the radish cruciferin gene, all three of which allow expression of the protein of interest in the seeds (Roberts et al., 1989; Anderson O. D. et al., 1989; Depigny-This et al., 1992).

In accordance with the invention, elements such as activators and introns may also be inserted into the expression cassette with the aim of amplifying the expression of the gene of interest.

An example of an activator is the translation activator of the TEV virus (Tobbaco Etch Virus) described by Carrington and Freed (1990). Among the introns which can be used, is the first intron of the maize Adh1S gene, which can be placed between the promoter and the encoding sequence. This intron, when it is included in a genetic construct, increases expression of the desired protein in maize cells (Callis et al., 1987).

It is also possible to use the first intron of the maize shrunken 1 gene (Maas et al., 1991), the first intron of the pea catalase gene, CAT-1 (Ohta et al., 1990), the second intron of the potato ST-LS1 gene (Vancanneyt et al., 1990), the intron of the TobYDV gene of the yellow dwarf virus (Morris et al., 1992), the intron of the Act 1 gene encoding rice actin 1 (Mc Elroy et al., 1990) or else intron 1 of the gene encoding triosephosphate isomerase (Snowdon et al., 1996).

Advantageously, the expression cassette may also contain 5′ untranslated sequences termed “leader sequences”. Such sequences can increase translation.

Among those known to those skilled in the art, mention may be made of:

the EMCV leader (EncephaloMyoCarditis virus 5′ noncoding region) (Elroy-Stein et al., 1989),

the TEV leader (Tobacco Etch Virus) (Carrington and Freed, 1990),

the leader of the BiP gene encoding human immunoglobulin heavy chain-binding protein (Macejack et al., 1991),

the AMV RNA 4 leader originating from the mRNA of the alfalfa mosaic virus protein (Jobling et al., 1987),

the leader of the tobacco mosaic virus (Gallie et al., 1989).

Among the terminators which may be used in the constructs of the invention, mention may in particular be made of:

the 35S polyA of the cauliflower mosaic virus (CaMV), described in the article by Franck et al., (1980),

the nos terminator corresponding to the 3′ noncoding region of the nopaline synthase gene of the Agrobacterium tumefaciens Ti plasmid (Depicker et al., 1992),

the histone gene terminator (EP 0 633 317).

According to a preferred embodiment, the transcription terminator is the nos terminator of the Agrobacterium tumefaciens nopalin synthase gene (Depicker et al., 1992) or else the 35S polyA sequence of the cauliflower mosaic virus (CaMV), described in the article by Franck et al., (1980).

In a variant of the implementation of the invention, said regulatory sequences also comprise addressing signals capable of directing the expression specifically in a particular type of cellular compartment, for example the extracellular space or the apoplasm.

As an example of chloroplast addressing signals, mention may be made of the sequence encoding the transit peptide of the precursor of the ribulose-1,5-bisphosphate carboxylase oxygenase small subunit from Pisum sativum. As mitochondrial addressing signals, mention may be made of the sequence encoding the transit peptide of the precursor of the mitochondrial ATPase F1 β-subunit from Nicotiana plumbaginifolia.

These transit peptides, comprising the N-terminal methionin, are normally cleaved in the chloroplasts or mitochondria. Expression of proteins in these organelles therefore also has the characteristic of producing a molecule lacking the N-terminal methionine like the natural molecule.

According to another variant, the addressing sequences may be sequences encoding a N-terminal signal peptide (“prepeptide”), optionally in combination with a signal responsible for retention of the protein in the endoplasmic reticulum (signal of the KDEL or KTEL type), or a vacuolar addressing signal or “propeptide”.

The presence of the N-terminal signal peptide or prepeptide allows the neopolypeptide to be introduced into the endoplasmic reticulum, where a certain number of posttranslational modifications take place, in particular cleavage of the signal peptide, N-glycosylation, if the protein in question has the N-glycosylation sites, and formation of disulfide bridges. Among these various signals, the prepeptide, which is responsible for addressing the protein into the endoplasmic reticulum, is very useful. It is normally a hydrophobic N-terminal signal peptide which has between 10 and 40 amino acids and is of animal or plant origin. Preferably, it is a prepeptide of plant origin, for example that of sporamin, of barley lectin, or plant extensin, or of the PR1 or PR2 protein.

Normally, the signal peptide is cleaved by a signal peptidase as soon as co-translational introduction of the neopolypeptide into the lumen of the RER (rough endoplasmic reticulum) takes place. The mature protein no longer comprises this N-terminal extension.

To obtain secretion, according to one embodiment of the invention, it is possible to use the signal peptide of sporamin A of sweet potato tuberous roots.

The addressing sequences may, besides the prepeptide, also comprise an endoplasmic retention signal, consisting of the KDEL, KTEL, SEKDEL or HEKDEL peptides. These signals are normally found at the C-terminal end of the protein and may remain on the mature protein. The presence of this signal tends to increase the yields of recombinant proteins.

The presence of sequences of the KDEL or KTEL type at the C-terminal end of proteins leads to the retention thereof in the endoplasmic reticulum and therefore to cellular compartmentalization of these proteins. This compartmentalization may, in certain cases, block the accessibility of the enzyme to its substrate present in a different cellular compartment, leading to inactivity of the protein. Thus, according to one variant, it is possible to envision the elimination of this type of addressing sequence without losing the enzymatic activity of the protein. The elimination of these sequences may lead to apoplastic expression of the protein, possibly allowing better accessibility of the protein to its substrate. The elimination may also lead to expression of the protein in a compartment other than the one in which its substrate is expressed, thus avoiding rapid degradation of the phytic acid liable to impair the development of the seed.

The activity of the enzyme with respect to its substrate can therefore be controlled in space and in time. A subsequent step of grinding the seed can optionally be used to allow access of the enzyme to its substrate.

The elimination of the KTEL or KDEL sequence can in particular be carried out by the PCR amplification technique, using primers specific for the C-terminal portion of the protein just upstream of the KTEL or KDEL sequence and adding a stop codon to the primer.

The addressing signals may, besides the prepeptide, also comprise a vacuolar addressing signal or “propeptide”. In the presence of such a signal, after passing through the RER, the protein is addressed to the vacuoles of the aqueous tissues, for example the leaves, and also to the protein bodies of the storage tissues, for example the seeds, tubers and roots.

Addressing of the protein to the protein bodies of the seed is particularly advantageous because of the ability of the seed to accumulate proteins, up to 40% of proteins relative to solids, in cellular organelles derived from vacuoles, called the protein bodies, and because of the possibility of storing the seeds containing the recombinant proteins in the dehydrated state, for several years.

As a propeptide, use may be made of a signal of animal or plant origin, plant signals being particularly preferred, for example pro-sporamin. The propeptide may be N-terminal (“N-terminal targeting peptide” or NTTP) or C-terminal (CTTP). Insofar as the propeptides are normally cleaved as soon as the protein enters the vacuole, they are not present in the mature protein.

The use of the signal peptide or prepeptide can lead to the glycosylation of the protein.

In the absence of any addressing signal, the protein is expressed in the cytoplasm.

The invention also relates to a vector into which the expression cassette as defined is inserted. This vector may be a plasmid which may also comprise a marker gene for distinguishing a transformed plant from a plant which does not contain the foreign DNA transferred. Thus, in accordance with the invention, the vector may include, as a marker gene, both selectable genes which confer resistance to an antibiotic or to a herbicide, and reporter genes. Among the selectable genes which can be used, mention may be made of:

the sul gene which confers resistance to the sulfonamide herbicide Asulam (WO 98/49316),

the nptII gene which confers resistance to kanamycin (Bevan et al., 1983),

the hph gene which confers resistance to hygromycin (Herrera-Estrella et al., 1983),

the bar gene which confers tolerance to bialaphos (White et al., 1990),

the EPSPS gene which confers tolerance to glyphosate (U.S. Pat. No. 5,188,642),

the HPPD gene which confers tolerance to isoxazoles (WO 96/38567),

the chloramphenicol acetyltransferase (CAT) gene which detoxifies chloramphenicol.

Similarly, mention may be made, as reporter genes, of:

the gene encoding the β-glucuronidase (GUS) enzyme, the gene of green fluorescent protein (GFP), which makes it possible to visualize the transformed cells under UV.

In accordance with the invention, as cloning or expression vectors comprising the fragment, mention may be made of the vectors comprising a DNA sequence containing at least one origin of replication, such as plasmids, cosmids, bacteriophages, viruses, etc. Plasmids are, however, preferred.

The invention also relates to the cellular, in particular bacterial, hosts containing the vectors mentioned above.

The invention also provides a method for producing transgenic plants, comprising, inter alia, the steps of:

transforming plant cells with an expression vector containing a nucleic acid fragment of the present invention,

selecting the transformed cells,

generating the transformed plants from these cells, expressing the inserted nucleic acid fragment.

The transformation of the plants according to the invention can be carried out by various methods, known to those skilled in the art. Mention should be made, for example, of methods of direct gene transfer, such as microinjection into cells of the plant embryo (Neuhaus et al., 1987) or electroporation (Chupeau et al., 1989), direct precipitation with polyethylene glycol (Schocher et al., 1986) or bombardment with a particle gun (Mc Cabe et al., 1988).

The plant can also be infected with a bacterial strain, in particular an Agrobacterium tumefaciens strain, according to a proven method (Schell and Van Montagu, 1983), or an Agrobacterium rhizogenes strain, in particular for species refractory to transformation (Chilton et al., 1982). The bacterial strain may comprise the gene encoding the plant MIPP enzyme, under the control of elements which provide expression of said gene. The bacterial strains may be transformed with a vector into which is inserted a sequence encoding said enzyme under the control of elements which provide expression thereof. This sequence is inserted, for example, into a binary vector such as pBIN19 (Bevan, 1984) or pMON 505 (Horsch and Klee, 1986), or any other binary vector derived from the pTi and pRi plasmids. It may be profitably introduced into the disarmed pTi or pRI plasmid, such as pGV 3850 (Zambryski et al., 1983), by homologous recombination, before transformation of the plant.

It has recently been shown that monocotyledonous plants such as rice and maize can be advantageously transformed with Agrobacterium tumefaciens (Hiei et al., 1994, Ishida et al., 1996).

According to a preferred method of the invention, the nucleic acid fragments are introduced into the cells by bombardment with particles covered with said fragments.

Particle bombardment offers the advantage of rapid transformation. Generally, immature embryos undergo only one bombardment. However, repeat bombardment may increase the frequency of transformation (WO 98/49316).

After the transformation step, the transformed cells are selected according to the phenotypic markers used in the vector. This selection is carried out on a medium containing a suitable selective agent. The transformed cells thus selected are then cultured and the plants are regenerated. DNA extraction and Southern blotting with probes specific for the gene of interest make it possible to confirm transformation. The methods for isolating DNA from biological material in culture and for verifying the presence of the insert are well known to those skilled in the art; they are, inter alia, described by Southern et al., 1975 and Mullis et al., 1987.

A subject of the present invention is more particularly a method for increasing the phytase activity of a plant, characterized in that overexpression of a MIPP, more particularly from maize, from rice or from wheat, is induced in said plant using an expression cassette as previously described and according to the preceding method.

The term “overexpression” is here intended to mean both an increase in the amount of MIPP compared to the amount expressed in a normal plant, and an ectopic expression of this enzyme, in a tissue or a compartment and/or at a developmental stage where it is not normally expressed.

In accordance with the present invention, the foreign sequence may be heterologous, i.e. it comes from a plant different from the host cell. It may also be a sequence of the MIPP naturally produced by the plant.

A subject of the present invention is also plant hosts, consisting of plant or plant organs, transformed with one or more nucleic acids of the invention and according to the method defined above. If the transformation involves several genes of interest, then these genes may be inserted into the same expression cassette or else into different expression cassettes.

The term “plant hosts” encompasses both the plants and the plant cells or the various parts of the plant, such as seed, fruit or leaf.

As examples of transgenic plants according to the invention, mention may be made of cereals, in particular maize, wheat, barley, sorghum, rye and rice, preferentially maize, and also pea, soybean and potato.

These transgenic plants of the invention encompass both the first generation plants and their descendants (lines or hybrids, in particular).

A subject of the present invention is more particularly transgenic plant hosts in which a plant MIPP, more particularly from maize, from wheat or from rice, can be expressed in a part of the plant or a cellular compartment where this enzyme is not naturally produced or is produced only in small amounts. These plant hosts are characterized by a genome in which an expression cassette according to the invention comprising regulatory sequences which induce specific expression in said part of the plant or said cellular compartment has been introduced. The plant host in question is advantageously a cereal, and even more advantageously maize, wheat and rice, or organs thereof.

The present invention relates more specifically to a transgenic plant according to the invention, characterized in that it produces transgenic seeds expressing an increased amount of enzymes with phytase activity.

The present invention also comprises the seeds of this transgenic plant, and in particular the seeds comprising an increased part of plant MIPP, obtained by specific expression of one of the nucleic acid sequences according to the present invention, in the seed.

Moreover, the flour and any product liable to be obtained from this seed are also part of the invention.

Finally, a subject of the present invention is a composition for human or animal nutrition comprising seeds, a seed flour or a plant MIPP, produced according to the present invention.

According to one embodiment, the nucleic acids defined by the invention can be used as a marker of the phenotype associated with the expression of MIPP proteins.

The invention allows the use of the nucleotide fragments as molecular markers in cultivation programs, in particular in the selection of varieties expressing an MIPP protein or a plant transformed with a sequence encoding this MIPP.

According to the present invention, these sequences make it possible to obtain the genomic DNAs by methods known to those skilled in the art, for example by screening genomic libraries according to the indications of Sambrook et al. (1989, Molecular cloning: a laboratory manual, Cold Spring Harbor Laboratory Press, New York). Similarly, using degenerate probes, they make it possible to identify genes encoding proteins homologous to the plant MIPP.

The transgenic plants expressing the plant MIPP according to the invention or the enzyme itself may be used for varied purposes, in any situation in which the phytase activity of this enzyme is necessary or desired.

The plant MIPP is thus of use in food preparation processes or in starch extraction processes (steeping process). For example, with this enzyme, it is possible to increase the extractibility of starches from the grain in which they are expressed. The phytate causes the proteins to precipitate with the starch, such that, if the amount of phytate is reduced, the extraction of the starch is consequently facilitated. Adding an enzyme with phytase activity to the grains of plants from which it is desired to extract the starch makes it possible to overcome the precipitation of the proteins by the phytates.

A plant transformed with a sequence encoding a plant MIPP according to the method defined by the invention, or a part of this plant, can be used to improve the digestibility of phytate, in the food intake of a monogastric animal, and consequently to decrease the amount of phytate in the solid manure or liquid manure.

The phytase activity of the plant MIPP can also be exploited to enhance the value of recovered steeping water or mashing water, in food preparations. In particular, the increased availability of phosphorus makes these waters very useful as additives for culture media or fermentation supports.

In addition, the phytase activity generated allows better recovery of inositol and derivatives thereof from steeping or mashing water or steeping water.

The invention also relates to the use of a plant MIPP according to the invention or the use of all or part of plants containing an MIPP according to the invention, for therapeutic or dietetic purposes, in particular for producing myoinositol and/or for decreasing phytic acid.

Phytic acid has been held responsible for certain types of impact on human health such as bone malformation, osteoporosis and anemia caused by iron deficiencies, or interference with assimilation of calcium, magnesium and zinc (Berlyne et al., 1973; Shan et al., 1979).

Myoinositol, the active nutritional form of inositol, is a constituent of the phospholipid phosphatidyl-inositol.

It is known for its virtues in the health domain. It has often been attributed with effects on decreasing the concentration of triglycerides and of cholesterol in the blood, and more generally for protection against cardiovascular diseases. In addition it is acknowledged that compounds derived from a phospholipid such as myoinositol have beneficial effects on insomnia and anxiety. Moreover, peripheral neuropathy in diabetics is one of the most paralyzing complications of diabetes. Now, it has been presumed for several years already that a decrease in the level of myoinositol is associated with the nerve fiber damage in diabetics suffering from such a complication.

Finally, the invention relates to the use of the plant MIPP according to the invention, for releasing the inorganic phosphate directly assimilable by monogastrics (Pointillard, 1994) and/or improving the availability of the cations chelated by phytin, such as iron, calcium, magnesium or zinc. This avoids adding supplements to animal food intakes and makes it possible to increase the nutritive value of the seed.

### LEGENDS OF THE FIGURES

FIG. 1 represents an alignment of the nucleic acid sequences encoding the maize and rice MIPPs.

FIG. 2 represents an alignment of the protein sequences of rice and maize MIPP.

FIG. 3 represents a comparison of the amino acid sequences of rice and maize MIPPs with animal MIPPs, acid phosphatases and fungal phytases.

FIG. 4 is a restriction map of the plasmid pRD-257.

FIG. 5 is a restriction map of the plasmid p3214.

FIG. 6 is a restriction map of the plasmid PBIOS 421.

FIG. 7 is a restriction map of the plasmid pBIOS 422.

### SEQUENCE LISTING

SEQ ID No. 1: Nucleic acid sequence of maize isolated cDNA.

SEQ ID No. 2: Amino acid sequence of a maize MIPP protein (ZmMIPP).

SEQ ID No. 3: Nucleic acid sequence of rice isolated cDNA.

SEQ ID No. 4: Amino acid sequence of a rice MIPP protein (OsMIPP).

SEQ ID No. 5: Oligonucleotide 5′olZMP1 used as a 5′ primer to obtain the ZmMIPP cDNA.

SEQ ID No. 6: Oligonucleotide 3′olZMP used as a 3′ primer to obtain a ZmMIPP cDNA.

SEQ ID No. 7: Internal oligonucleotide olMIPP1.

SEQ ID No. 8: Internal oligonucleotide olMIPP2.

SEQ ID No. 9: Oligonucleotide 5′olOSp used as a 5′ primer to obtain the OsMIPP cDNA.

SEQ ID No. 10: Oligonucleotide 3′olOSp used as a 3′ primer to obtain the OsMIPP cDNA.

SEQ ID No. 11: Oligonucleotide 5′olSOP1 used as a 5′ primer to obtain the OsMIPP cDNA.

SEQ ID No. 12: Oligonucleotide olMIPP9 used as a 3′-primer to obtain the cDNA encoding a wheat MIPP.

SEQ ID No. 13: Oligonucleotide olMIPP10 used as a 3′ primer to obtain the cDNA encoding a wheat MIPP.

SEQ ID No. 14: Nucleic acid sequence of the EST 3′TaMIPP.

SEQ ID No. 15: Putative signal peptide.

SEQ ID No. 16: Putative signal peptide.

SEQ ID No. 17: Nucleic acid sequence of maize isolated cDNA.

SEQ ID No. 18: Amino acid sequence of a maize MIPP protein (ZmMIPP).

### EXAMPLES

### Example 1

### Isolation of the cDNA Clones Encoding a Plant MIPP Protein

The sequences of plant MIPPs can be obtained by carrying out a bioanalysis study using sequences encoding animal MIPPs, presented by Caffrey et al. (1999). These sequences thus make it possible to define homologies with referenced cDNA sequences, for example, in databanks specific to maize, to wheat and to rice.

By identifying the ESTs specific to the sequences homologous to said animal sequences, oligonucleotides can be defined and used as primers to isolate these cDNAs of interest from cDNA or cDNA libraries, by PCR.

1.1—Maize MIPP (ZMMIPP)

1.1.1—Choice of the Variety

Two Zea Mays lines were used: the B73 line and the HiII hybrid (one of its parents being B73). Total RNA extraction was carried out on immature leaves for the B73 line and on calluses for the HII hybrid (Armstrong et al., 1991).

Culture Conditions

Immature Leaves

40 B73 seeds are put into compost and placed in a greenhouse. The immature leaves are harvested once the stage at which 7-8 leaves are visible has been reached, i.e. approximately 3-4 weeks in a greenhouse. The immature leaves are still in the cornet. A sample was taken at 3 weeks and at 4 weeks.

Immature leaves were also taken at 2 weeks after germination.

Obtaining Type II Calluses

The ears are sampled when immature embryos have reached a size of 1.5 mm to 2 mm, i.e. 10 days after fertilization. The spathes and the bristles are removed from the harvested ears, which are then disinfected with 20% (v/v) Domestos® for 15 minutes with agitation.

The ears are rinsed three times with sterile water. The upper part of the grain is cut so as to uncover the albumen, and then a slight pressure on the grain makes it possible to free the albumen. The immature embryo which is still in the grain is extracted and then deposited onto the callogenesis medium N6P6 (3.98% (w/v) N6 salt (Sigma C1416); 5 ml/l vitamins. N6; 0.7% (w/v) L-proline; 0.1% (w/v) caseine hydrolysate; 20% (w/v) sucrose; 0.001% (w/v) 2,4-D; 2.5% (w/v) phytagel, pH 5.8), placing it flat side down onto the agar. The embryos are cultured for 15 days in a culture chamber at 26° C. and in the dark. The embryos are separated from their radical and are then transplanted onto a new N6P6 medium. The callus which proliferates is transplanted every 2 to 3 weeks onto N6P6 medium (dish of 16 calluses). The calluses are multiplied in a culture chamber at 26° C. and in the dark.

1.1.2—Total RNA Extraction and cDNA Synthesis

Total RNA extraction was carried out according to the method described by Verwoerd et al. (1989).

For each tissue, two total RNA extractions were carried out.

To synthesize the first cDNA strand, the directions given in the SMART PCR cDNA synthesis kit, marketed by Clontech, were followed.

1.1.3—Isolation of a Maize ZmMIPP cDNA Clone

The oligonucleotides used as PCR primers for the cDNA screening are given in the attached annex.

The oligonucleotides were determined from the nucleic acid sequences of the ESTs identified by bioanalysis as being strongly homologous to the sequences encoding animal MIPPs. The oligonucleotides which frame the coding sequence of the ZmMIPP gene possess restriction sites positioned 5′: NcoI or NdeI and positioned 3′: BamHI, this being to allow oriented cloning into the bacterial expression vector pET-14b (Novagen). The internal oligonucleotides olMIPP1 and olMIPP2 (SEQ ID No. 7 and 8), which are used to verify the identity of the sequences, do not have any.

PCR Amplification

The manipulation was repeated on two different pools of total RNA for the same species and the same tissue.

The amplification reaction was carried out directly on the first cDNA strand synthesized for the total RNA.

The pair of primers 5′olZMP1-3′olZMP (SEQ ID No. 5 and 6) on the cDNAs of immature leaves and calluses made it possible to amplify a 1.6 kb fragment. A PCR was carried out on this fragment, with the pairs of oligonucleotides 5′olZMP1-olMIPP2 (SEQ ID No. 5 and 8) and olMIPP1-3′olZMP (SEQ ID No. 7 and 6), olMIPP1 and olMIPP2 being internal oligonucleotides specific for the RHGXRXP consensus, generating respectively an amplification at 0.2 kb and 1.4 kb. This result shows the presence of the RHGXRXP site in the 1.6 kb fragment. Consequently, it was cloned into PGEM-T (Promega) and sequenced.

1.1.4—Deduction of the Amino Acid Sequence and Analysis

The nucleic acid sequence and the amino acid sequence are given in the attached annex, respectively by SEQ ID No. 1 and SEQ ID No. 2. They are valid for the immature leaf cDNA clone and the callus cDNA clone of maize. Specifically, sequencing showed that they are identical. Analysis of the nucleotide sequences carried out with the MAC VECTOR 6.5.3 program—Oxford Molecular.

Biochemical Characteristics of ZMMIPP (with the MAC VECTOR 6.5.3 Program—Oxford Molecular)

| calculated molecular mass | 58.6 kDa |
|---|---|
| pI | 8.95 |

With the Protein Motif Detection Programs (SEQWEB Version 1.1—Wisconsin Package Genetic Computer Group), Identification of:

a putative signal peptide of 25 amino acids at the N-terminal end of the protein: MGMAAPRAPLPLPQLLLLLVAALLA (SEQ ID No. 15)

a putative signal for retention in the endoplasmic reticulum at the C-terminal end of the protein: KTEL

1.2—Rice MIPP (OsMIPP)

1.2.1—Choice of Variety

The plant material is Oryza sativa japonica, variety Nipponbare. The RNA is extracted from rice calluses.

Culture Conditions:

Induction of rice calluses. The external envelopes are removed from the rice grains at maturity, which are disinfected for 1 min in 70% (v/v) ethanol and 30 min in 50% (v/v) domestos. The grains are rinsed in sterile distilled water and deposited onto N6P6 medium contained in Petri dishes. The dishes are sealed and placed in an incubator for 21 days, at 28° C. in the dark. A primary callus develops from the scutellum of the embryo. Around the primary callus, small embryogenic units individualize and are transferred into Petri dishes containing N6P6 medium and incubated for 14 days at 28° C. and in the dark. During this period, they will increase in size. The calluses are then taken for RNA extraction.

1.2.2—Total RNA Extraction and cDNA Synthesis

Total RNA extraction was carried out according to the method described by Verwoerd et al. (1989).

Two total RNA extractions from the calluses were carried out.

To synthesize the first cDNA strand, the directions given in the SMART PCR cDNA synthesis kit, marketed by Clontech, were followed.

1.2.3—Isolation of a Rice OsMIPP cDNA Clone

The oligonucleotides used were determined from the ESTs identified by bioanalysis as being strongly homologous to the sequences encoding animal MIPPs. The oligonucleotides which should frame the coding sequence of the MIPP gene have restriction sites positioned 5′ NcoI or NdeI and positioned 3′: BamHI, this being to allow oriented cloning into the bacterial expression vector pET-14b. The internal oligonucleotides which are used to verify the identity of the sequences do not have any.

PCR Amplification:

The manipulation was repeated on two different pools of total RNA for the same species and the same tissue.

The amplification reaction was carried out directly on the first cDNA strand synthesized from the total RNA.

The amplification reaction with the pairs of oligonucleotides 5′olOSP-3′olOSP (SEQ ID No. 9 and 10) or 5′olOSP1-3′olOSP (SEQ ID No. 11 and 10) on the callus cDNAs generates a 1.6 kb fragment. A PCR was carried out on this fragment with the pairs of oligonucleotides 5′olOSP1-olMIPP2 and olMIPP1-3′olOSP, olMIPP1 and olMIPP2 being internal oligonucleotides generating respectively amplification at 0.2 kb and 1.4 kb. This result shows the presence of the RHGXRXP site in the 1.6 kb fragment. Consequently, it was cloned into pGEM-T (Promega) and sequenced.

1.2.4—Deduction of the Amino Acid Sequence and Analysis

The nucleic acid sequence and the amino acid sequence are given in the attached annex, respectively by SEQ ID No. 3 and 4.

Biochemical Characteristics of OsMIPP:

| Calculated molecular mass | 56.5 kDa |
|---|---|
| pI | 8.3 |

With the protein motif detection programs, identification of:

a putative signal peptide of 18 amino acids at the N-terminal end of the protein: MAAPRTPLPLVLLLVSAA (SEQ ID No. 16)

a signal for retention in the endoplasmic reticulum at the C-terminal end of the protein: KSEL.

1.3—Analysis of the Protein Sequences Deduced from the OSMIPP and ZmMIPP cDNA clones

1.3.1—OsMIPP and ZMMIPP Comparison

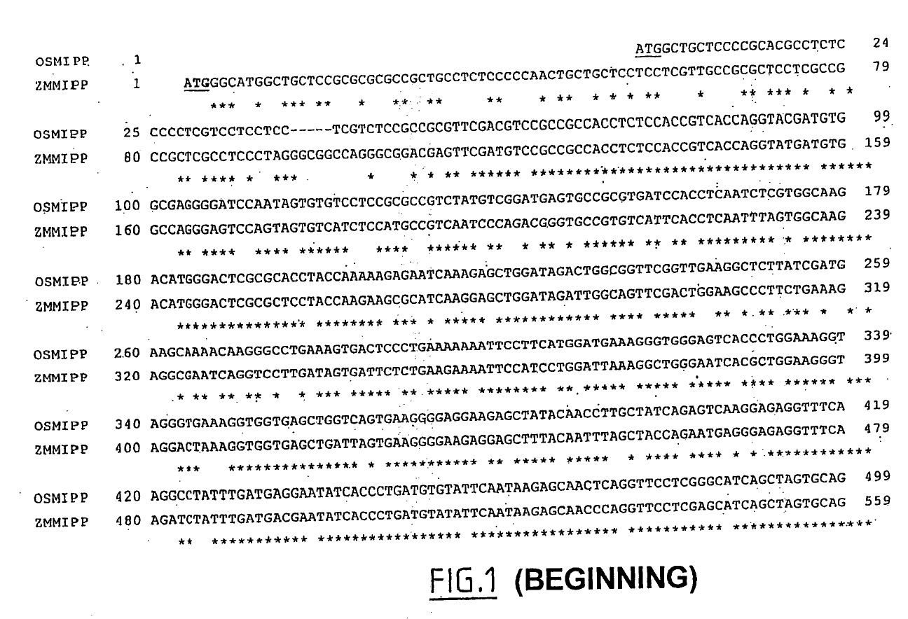

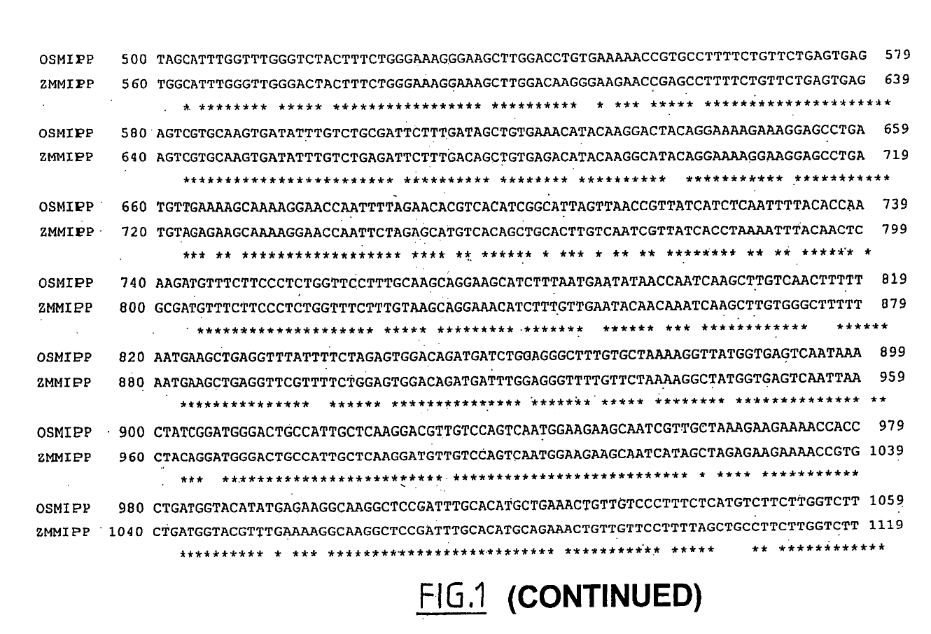

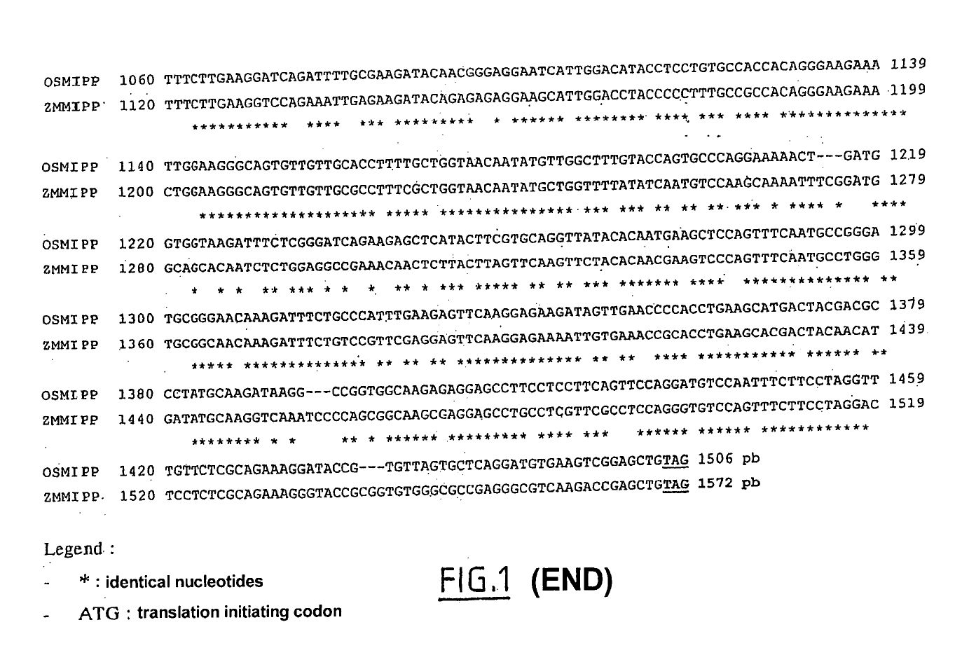

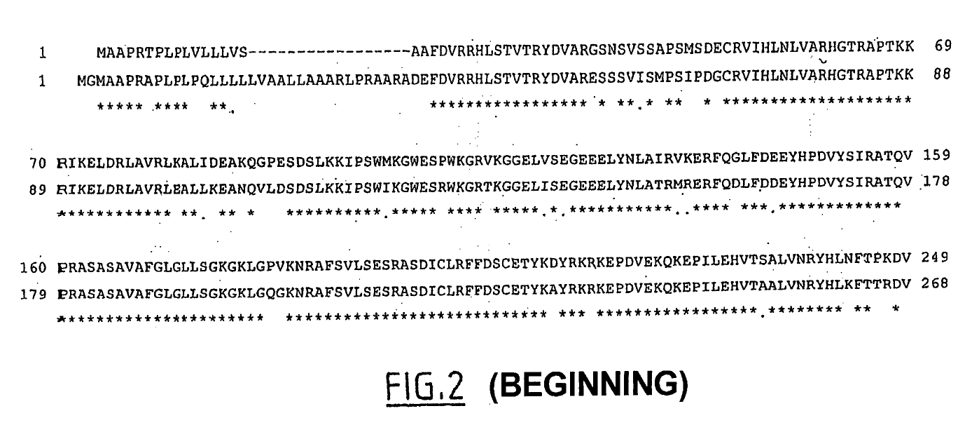

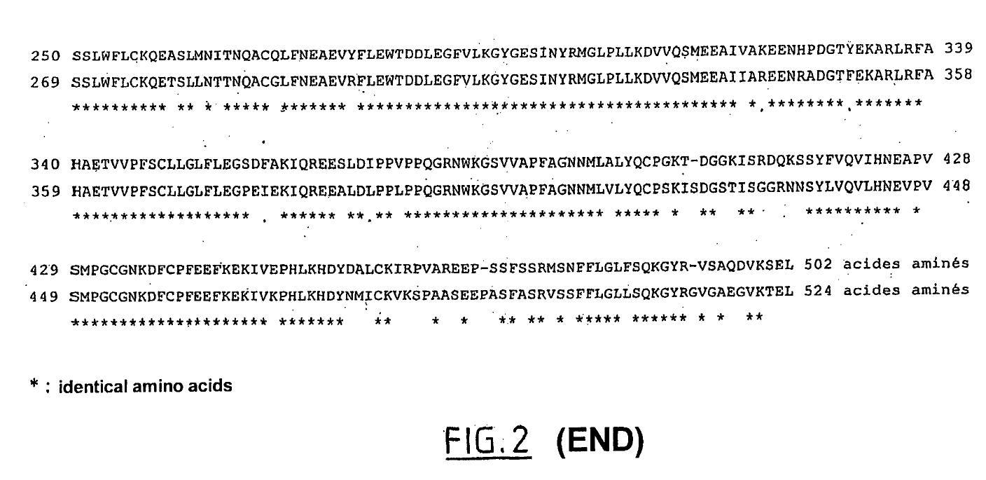

The protein sequences of the rice and maize MIPP proteins are strongly homologous to one another. The results of this alignment, obtained using the MAC VECTOR™ 6.5.3 program—Oxford Molecular, are given in the attached annex, FIG. 1.

1.3.2—Comparison Between OsMIPP/ZmMIPP and Acid Phosphatases

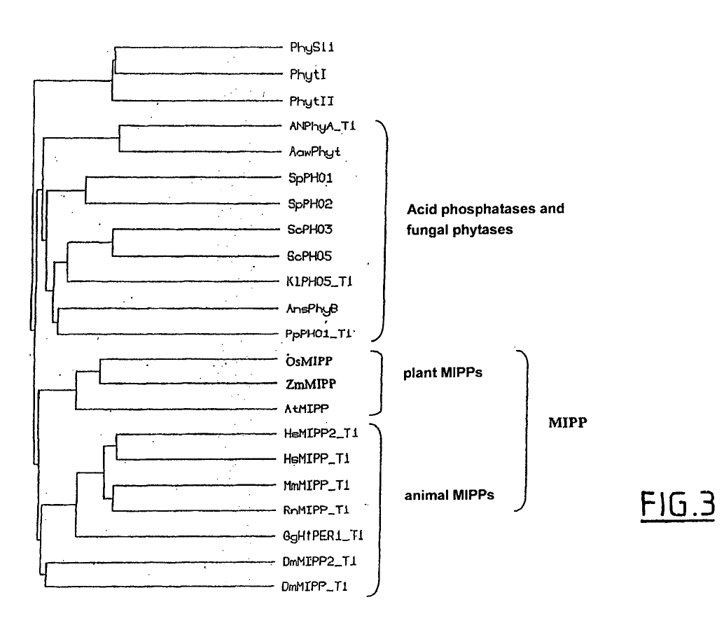

A bioanalysis of the protein sequences cloned via the cDNAs, as previously, demonstrates homology with two families of acid phosphatases: animal. MIPPs and fungal phytases. The results of this study are given in the attached annex, FIG. 3.

MIPPs are proteins described only in the animal domain.

These proteins hydrolyze inositol polyphosphate compounds, particularly tetra-, penta- and hexainositol phosphate, and consequently phytic acid (Craxton et al., 1997). This suggests that the maize and rice cDNAs thus cloned encode enzymes homologous to animal MIPPs, with in particular phytase activity.

1.4 Obtaining the cDNA Encoding a Wheat MIPP

Comparison of the biochemical characteristics of the rice and maize MIPPs with wheat phytase reveals similarities:

| | | | Wheat^{°} (Johansen |
|---|---|---|---|
| | Rice* | Maize* | et al., 1997) |
| Molecular mass kDa | 56.5 ± 2.5 | 58.6 ± 3.5 | 47 |
| pI | 8.3 | 8.95 | 7.4-8.2 |

*calculated values
^{°}apparent values.

In addition, it was noted that the plant MIPPs are homologous to animal MIPPs and to fungal phytases, proteins for which phytase activity is described.

These two observations show very clearly that the wheat grain bran phytase is a MIPP.

Grains of Triticum aestivum wheat at various developmental stages: 10, 20, 30 and 40 days after anthesis (DAA) were used as plant material.

In wheat, fertilization takes place in the flower which is still closed, and consequently it is not visible. When this has taken place, the anthers appear, flowering occurs. This step also corresponds to the end of anthesis or anther development. The days corresponding to the period of grain development begin from this moment.

The authors of the invention measured the kinetics of the phytase activity of a fraction of the grain enriched in bran during development at 10, 20, 30 and 40 DAA. This phytase activity increases during the development of the grain. This increase may be explained by activation of the enzymes and/or by accumulation of the enzymes in the bran during the development of the grain.

Total RNA extraction was then carried out for the 4 stages: 10, 20, 30 and 40 DAA. Expression kinetics, i.e. Northern blots, with the OsMP probe (nucleotide 105 to nucleotide 1506—sequence corresponding to 467 amino acids of the C-terminal portion of the protein) or the probe ZmMP (nucleotide 1 to nucleotide 1572-sequence corresponding to 524 amino acids of the protein), were produced on the total RNA extracted from grain bran taken at 10, 20, 30 and 40 DAA.

Moreover, the cDNAs corresponding to the total RNA extracted from bran taken at the four stages of development of the grain were synthesized.

The wheat MIPP cDNA is isolated by RT-PCR or by RACE-PCR.

The oligonucleotides used for the amplification reactions are determined on the nucleic acid sequences of the rice and maize MIPP cDNA clones, in the areas in which the sequence homologies are strongest. It is also possible to use degenerate oligonucleotides, determined from the nucleic acid sequences, in particular those of rice and of maize, corresponding to the N- and C-terminal regions of the plant MIPPs.

Moreover, the nucleic acid sequence of the OsMIPP cDNA was used to search for homologous sequences in Triticum aestivum wheat EST bases. An EST derived from wheat cDNA was selected; it is named 3′TaMIPP (SEQ ID No. 14). The protein sequence deduced from the EST exhibits strong homology with the 58 amino acids of the C-terminal portion of the OsMIPP protein. This EST was used to define two primers, olMIPP9 and olMIPP10 (SEQ ID No. 12 and SEQ ID No. 13). These primers, combined with the Smart II oligonucleotide™ primer of the SMART PCR cDNA synthesis kit (Clontech), makes it possible to isolate, by PCR, a cDNA encoding a wheat MIPP. The PCR generates various fragments., which are separated in agarose gel, transferred onto a nylon membrane and hybridized with the probe ZMMP. The fragments recognized by the probe ZMMP are homologous to ZmMIPP.

### Example 2

### Production of the ZmMIPP and OsMIPP Proteins in E. coli and Evaluation of the Phytase Activity of the Recombinant Proteins

2.1—Cloning into the Expression Vector pET-14b

The NdeI restriction site, introduced by the oligonucleotides 5′olZMP1 and 5′olOSP1, and the BamHI restriction site, introduced by the oligonucleotides 3′olZMP1 and 3′olOSP1, frame the ZMMIPP and OsMIPP cDNAs. They allow oriented and in-phase cloning of the cDNAs at the NdeI and BamHI restriction sites of the bacterial expression vector pET-14b (Novagen). The vectors obtained, pET-OsMIPP and pET-ZmMIPP, were verified by sequencing.

2.2—MIPP Production in E. coli

The protein production is carried out according to the recommendations of the supplier Novagen. E. coli strain BL21(DE3)pLysS bacteria were used. The vector pLysS makes it possible to avoid expression of the recombinant protein where production has not been induced and facilitates cell lysis since it carries the gene encoding the T7 phage lysozyme.

E. coli strain BL21(DE3)pLysS bacteria are transformed with the vectors pET-OsMIPP and pET-ZmMIPP; these bacterial transformants are used, respectively, to produce the OsMIPP and ZmMIPP proteins. E. coli strain BL21(DE3)pLysS bacteria are transformed with pETE-14b; these bacterial transformants are used as a negative control for the production. E. coli strain BL21(DE3)pLysS bacteria are also used as a negative control for: production.

Each bacterial transformant is cultured in 5 ml of LB medium (Luria-Bertani medium) in the presence of suitable antibiotics at 37° C. with shaking (175 rpm) for 15 hours. Two antibiotics are used: carbenicillin and chloramphenicol. The chloramphenicol resistance is provided by the vector pLysS (concentration used: 30 μg/ml). The carbenicillin resistance is provided by the vector pET-14b (concentration used: 50 μg/ml). The appropriate use of one or both antibiotics makes it possible to select the bacterial transformants which contain the vectors pLysS and/or pET-14b. A volume of 1 ml of each preculture is used to feed a volume of 100 ml of LB medium with the appropriate antibiotics, contained in a 500 ml Erlenmeyer flask. The cultures are incubated at 37° C., with shaking (175 rpm), until the optical density at 600 nm is OD₆₀₀ₙₘ=0.6. When the OD₆₀₀ nm=0.6: the 100 ml of LB medium are distributed equally into two 250 ml Erlenmeyer flasks. The first is used for protein production. Production is induced via the 500 μl of 100 mM IPTG, i.e. a final concentration of 1 mM. Production is carried out for 6 h at 30° C. with shaking (175 rpm). The second Erlenmeyer flask is used as a noninduced control. It is incubated for 6 h at 30° C. with shaking (175 rpm).

After production, the bacterial culture is centrifuged at 3000 rpm for 5 min at 4° C. The bacterial pellet is taken up in a volume of TE buffer, pH 8.0 (10 mM Tris-HCl, 1 mM EDTA), such that the TE volume/culture volume ratio is 1/20. The bacteria are stored at −80° C. The bacteria are lyzed in two steps a freeze-thawing step (two cycles of freezing at −80° C./thawing at 37° C.), an ultrasound step: 3 times 15 s, 30%, 4 pulses/s.

Between each cycle, the sample is placed in ice. A cocktail of protease inhibitors is added to the bacterial lysate, which contains the bacterial proteins and the heterologous proteins, in order to preserve them.

2.3—Protein Assay

The total protein concentration of the extracts is evaluated according to the Bradford method (1976). 2 μl of bacterial lysate are added to 200 μl of Coomassie reagent (Bio-Rad protein assay), in a microtitration plate. For the negative control, the protein extract is replaced with 2 μl of TE buffer, pH 8.0. After homogenization, the absorbances are read at 595 nm with a software (Labsystems)-assisted fluorimeter (Labsystems, Genesis V2.00). The amount of proteins is determined from a calibration curve produced with bovine serum albumin (1 mg/ml).

2.4—Analysis of the Protein Extracts by Polyacrylamide Gel Electrophoresis in the Presence of Sodium Dodecyl Sulfate (SDS-PAGE)

This was carried out according to the Laemmli method (1970) according to the instructions for Bio-Rad.

The separating gel comprises 10% polyacrylamide (29/1 acrylamide/bisacrylamide, w/v) containing 0.38 M Tris-HCl, pH 8.8 and 0.1% SDS (w/v). The stacking gel comprises 4% polyacrylamide containing 0.12 M Tris-HCl, pH 6.8 and 0.1% SDS (w/v). Polymerization of the gel occurs in the presence of 0.05% (w/v) ammonium persulfate and 0.078% (v/v) Temed. A 40 μg amount of proteins is used for the SDS-PAGE analysis. The proteins are taken up in the loading buffer composed of 30 mM Tris-HCl, pH 6.8, 12.5% (v/v) glycerol, 1% (w/v) SDS and 0.005% (w/v) bromophenol blue, in the presence of 100 mM dithiothreitol and/or 360 2-mercaptoethanol.

The samples are incubated at 100° C. for 5 minutes before being loaded onto the gel. BENCHMARK prestained protein ladder proteins (Gibco BRL) are used as molecular mass markers. The migration buffer is composed of 25 mM Tris-base, 0.2 M glycine and 0.1% (w/v) SDS. The electrophoresis is carried out at a constant voltage of 150 volts for 1 hour at ambient temperature. After migration, the gel is rinsed three times for 10 minutes in water, the proteins are stained with a solution of Coomassie blue G250 (Bio-Safe Coomassie, Bio-Rad) for 1 hour, and the gel is then rinsed in water, at ambient temperature.

The SDS-PAGE analysis was carried out on crude bacterial extracts. The presence of the heterologous protein in the bacterial culture after induction of production is difficult to determine. An analysis by Western blotting with a suitable antibody makes it possible to confirm the presence of the heterologous protein. Specifically, in this case, the heterologous protein has the advantage of being synthesized with a peptide at its N-terminal end. Antibodies specific for this peptide are commercially available and can be used to demonstrate the heterologous protein. This is along the lines of the study by Craxton et al. (1997): in tests for production of an animal MIPP in E. coli, the protein could only be detected by immunodetection.

2.5—Evaluation of the Phytase Activity

The phytase activity is determined by assaying phosphate released by the enzyme from sodium phytate.

The phytate activity is measured directly on bacterial lysates. 75 μl of bacterial lysate are mixed, in a 1.5 ml Eppendorf tube, with 300 μl of solution containing 5 mM sodium phytate, 0.25 M sodium acetate, pH 4.8, and 1 mM CaCl₂. The enzymatic reaction takes place for 1 hour at 55° C. For each assay, an identical control is kept at 0° C. during incubation. This control, used as a zero, makes it possible to eliminate the possible absorbances due to the protein extracts themselves. The enzymatic reaction is stopped by adding 375 μl of 20% (w/v) trichloroacetic acid, followed by centrifugation at 8000 rpm for 15 minutes and at 4° C., which makes it possible to precipitate the protein. The free phosphate is assayed on the supernatant. 750 μl of the following reagent: 0.38 M FeSO₄, 0.16 N H₂SO₄/12 mM ammonium molybdate, 1 N H₂SO₄, 1/4, v/v, are added to 750 μl of supernatant. The absorbance is measured at 690 nm. The amount of phosphate is determined from a standard curve. The phytase activity is expressed in nmole of phosphate released per hour, per mg of total protein at 55° C.

The phytase activity was effected on bacterial extracts. A step of purification of the heterologous protein from the crude bacterial extracts is, however, preferable in order to improve the interpretation of the results. This can be readily envisioned by virtue of the presence of the peptide at the N-terminal end of the heterologous protein, which makes it possible to purify by affinity chromatography.

### Example 3

### Construction of Chimeric Genes

3.1 For Constitutive Ectopic Expression of OsMIPP

To obtain constitutive expression, the pCsVMV promoter (Verdaguer et al., 1996, 1998) of the cassaya vein mosaic virus is used.

The construct is produced in the following way:

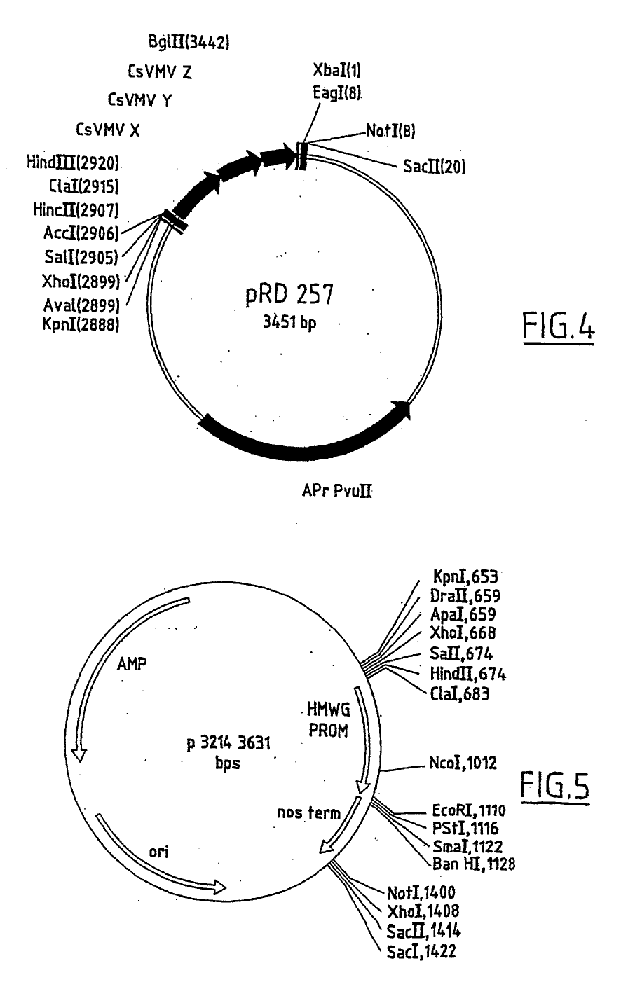

The 556 pb ClaI-SacII fragment of the vector pRD 257 (FIG. 4) contains the pCsVMV promoter. This fragment is cloned into the vector p3214 (FIG. 5) digested with ClaI and EcoRI. The vector obtained is named vector pBIOS 366. It contains the pCsVMV promoter and the ter nos terminator.

The OsMIPP cDNA was cloned into the vector pGEM-T (Promega), giving the vector pBIOS 367. The vector pBIOS 367 is digested with NotI, generating a 1563 pb fragment containing OsMIPP cDNA. This fragment is cloned into the vector PBIOS 366 digested with PstI. The vector PBIOS 368 is obtained, which contains the cassette: pCsVMV promoter, OsMIPP cDNA, ter nos terminator.

The vector pBIOS 368 is digested with XhoI, which makes it possible to remove the cassette: pCsVMV promdoter, OSMIPP cDNA, ter nos terminator. It is clone at the XhoI site of the binary vector PBIOS 273. The vector pBIOS 273 contains the Arabidopsis thaliana T-DNA, which contains the pAct1 promoter with the first intron of the rice Act1 gene, the coding sequence of the bar gene and nos terminator.

The vector obtained is the vector pBIOS 369. The vector pBIOS 369 comprises, firstly, the T-DNA which contains the expression cassette for the OsMIPP gene, inserted between the pCsVMV promoter and the Nos terminator, the expression cassette for the selectable gene, the Bar gene inserted between the pAct1 promoter and the first intron of the rice Act1 gene, and the nos terminator. In addition, PBIOS 369 also contains the gene for resistance to spectinomycin and an origin of replication which is functional in E. coli

3.2—For Tissue-Specific Ectopic Expression of OsMIPP

To obtain albumen-specific expression, the PHMWG promoter of the gene encoding a wheat high molecular weight glutenin is used.

The construct allowing expression of OSMIPP in the cytoplasm of the cells of the albumen is produced in the following way:

Digestion of the pBIOS 367 with the enzyme EcoRI generates a 1547 bp fragment containing the complete OSMIPP cDNA. This fragment is cloned at the EcoRI site of the vector p3214, between the PHMWG promoter and the ter nos terminator. The vector obtained is called vector pBIOS 370.

Digestion of the vector pBIOS 370 with the XhoI enzyme generates a 2287 pb fragment which is cloned at the XhoI site with the binary vector pBIOS 273. The vector obtained is named pBIOS 271. The vector PBIOS 371 comprises, firstly, the T-DNA which contains the expression cassette for the OsMIPP gene, inserted between the PHMWG promoter and the Nos terminator, the expression cassette for the selectable gene, the Bar gene inserted between the pAct1 promoter and the first intron of the rice Act1 gene, and the nos terminator. In addition, PBIOS 371 also contains the gene for resistance to spectinomycin and an origin of replication in E. coli.

3.3—For Albumen-Specific Ectopic Expression of ZmMiPP

To obtain albumen-specific expression, the PHMWG promoter of the gene encoding a wheat high molecular weight genome is used.

The construct allowing expression of ZmMIPP in the cells of the albumen is produced in the following way:

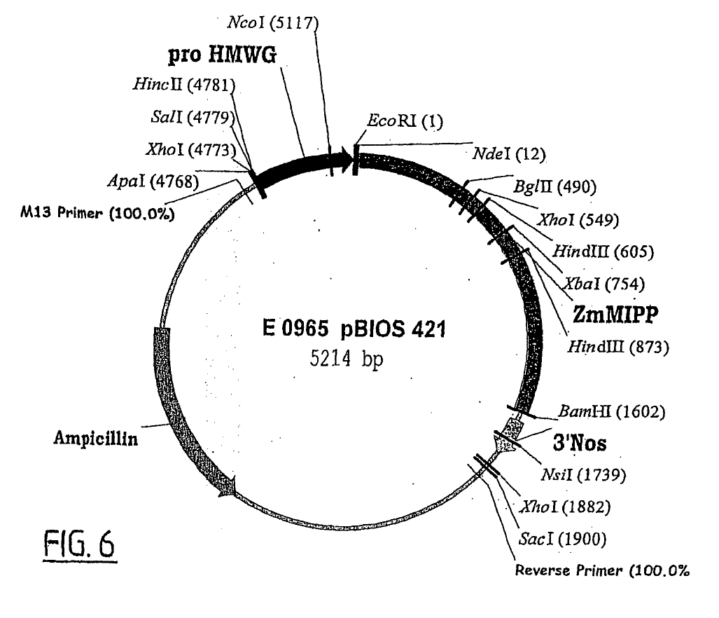

Digestion of the vector ZM4 (ZmMIPP fragment in the vector pGEM-T easy) with the EcoRI and BamHI enzymes generates a 1600 bp fragment containing the complete ZMMIPP cDNA. This fragment is cloned at the EcoRI and BamHI sites of the vector p3214 (FIG. 5), between the pHMWG promoter and the ter NOS terminator. The vector obtained is called vector pBIOS 421 and is described in FIG. 6.

Digestion of the vector pBIOS 421 with the SacI and SalI enzymes generates a 2335 pb fragment. The end of the 2335 pb fragment digested with the SacI enzyme is made blunt by treatment with the 3′-5′ exonuclease carried by the T4 DNA polymerase enzyme from New England Biolabs. This fragment is then cloned at the PmeI and XhoI sites of a binary vector derived from pSB12 (Japan Tobacco, EP 672 752) into which an expression cassette for the selectable gene described above has been inserted. The vector obtained is named pHMWG-ZmMIPP-JT. The vector pHMWG-ZmMIPP-JT comprises, firstly, the T-DNA containing the expression cassette for the ZmMIPP gene, inserted between the pHMWG promoter and the ter NOS terminator, the expression cassette for the selectable gene, the Bar gene (White et al., 1990) inserted between the pAct1 promoter and the rice Act1 first intron, and the ter NOS terminator. The expression cassette for the selectable gene is also inserted between the transposable elements Ac/Ds (Lechelt et al., 1989) which allow the selectable cassette to be excized after the action of a transposase. In addition, the vector pHMWG-ZmMIPP-JT also contains the gene for resistance to spectinomycin and an origin of replication in E. coli.

3.4—For Embryo-Specific Ectopic Expression of ZMMIPP

To obtain embryo-specific expression, and more particularly scutellum-specific expression, the pHyPRP promoter of the gene encoding a maize hybrid proline rich protein is used.

The construct allowing expression of the ZMMIPP in the cells of the embryo is produced in the following way:

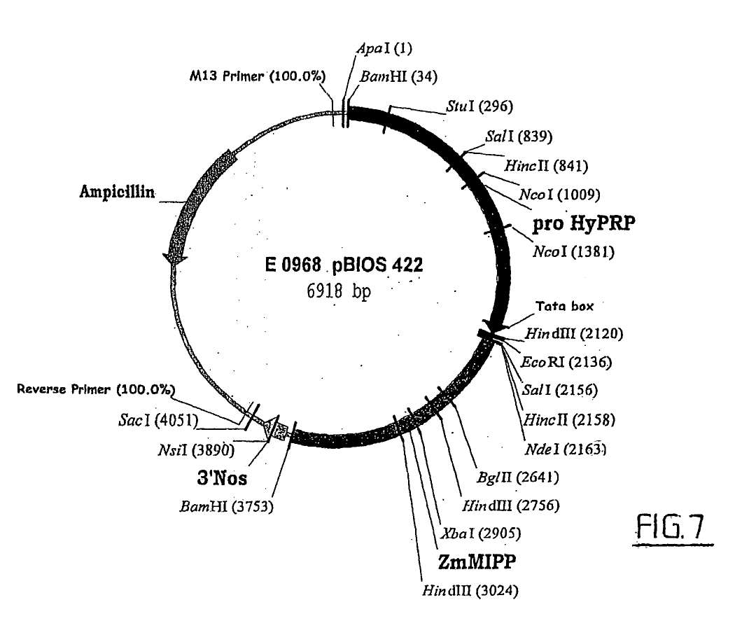

Digestion of the vector pBIOS 413 (pHyPRP fragment in the vector pBluescript) with the ApaI and NdeI enzymes generates a 2162 pb fragment containing the pHyPRP gene promoter described by Jose-Estanyol et al. (1992). This fragment is cloned at the ApaI and NdeI sites of the vector PBIOS 421 in front of the complete ZmMIPP cDNA and the ter NOS terminator. The vector obtained is called vector pBIOS 422, described in FIG. 7.

Digestion of vector pBIOS 422 with the SacI and ApaI enzymes generates a 4050 pb fragment. The ends of the 4050 pb fragment are made blunt by treatment with the 3′-5′ exonuclease carried by the T4 DNA polymerase enzyme from New England Biolabs. This fragment is then cloned at the PmeI and XhoI sites of the binary vector pBIOS 342, the ends of the vector being made blunt by treatment with the T4 DNA polymerase enzyme from New England Biolabs. This fragment is then cloned at the PmeI and XhoI sites of a vector derived from pSB112 (Japan Tobacco EP 672 752) as described in the preceding example. The vector obtained is named pHyPRP-ZmMIPP-JT. The vector pHyPRP-ZmMIPP-JT comprises, firstly, the T-DNA which contains the expression cassette for the ZmMIPP gene, inserted between the pHyPRP promoter and the ter NOS terminator, the expression cassette for the selectable gene, the Bar gene inserted between the pAct1 promoter and the rice Act1 first intron, and the ter NOS terminator. The expression cassette for the selectable gene is also inserted between the transposable elements Ac/Ds which make it possible to excise the selectable cassette after the action of a transposase. In addition, vector pHyPRP-ZmMIPP-JT also contains the gene for resistance of spectinomycin and an origin of replication in E. coli.

### Example 4

### Production of Transformed Maize

4.1—Transformation of Maize by Particle Bombardment

The genetic transformation of maize by particle bombardment is carried out on cells of embryogenic friable calluses or type II calluses. These calluses are obtained from immature embryos of genotype HII1 or A188×B73 according to the method described by Armstrong (1994). The calluses thus obtained can be multiplied and maintained by successive transplantations every two weeks, on the initiation medium. Plantlets are regenerated from these calluses by modifying the hormone and osmotic balance of the cells according to the method described by Vain et al. (1989).

The transfer of the genes of interest and selectable genes by particle bombardment into the type II calluses is carried out according to the following protocol.

Four hours before bombardment, fragments of type II calluses, with a surface area of 10 to 20 mm², are placed at the center of a Petri dish containing the initiation medium supplemented with 0.2 M mannitol and 0.2 M sorbitol, at 16 fragments per dish.

The vectors carrying the genes of interest and selectable genes are prepared using the CONCERT system according to the supplier's instructions (GIBCO BRL).

They are then precipitated on particles of tungsten (M10) according to the protocol described by Klein (1987). The particles thus coated are projected onto the target cells using a particle gun. Optimization of the bombardment conditions may depend on the type of device used and is included in the techniques normally mastered by those skilled in the art.

After bombardment, the dishes are sealed with Scellofrais® and placed in the dark at 27° C. The calluses are transferred onto an initiation medium supplemented with a selective agent, 24 h after bombardment.

The calluses are maintained on this medium for 3 months, and the medium is changed every 2 weeks. The calluses whose growth is not inhibited by the selective agent are usually and mainly composed of cells resulting from the division of a cell which has integrated into its genetic inheritance one or more copies of the selectable gene. The frequency of production of such calluses is approximately 0.8 calluses per dish bombarded.

These calluses are identified, individualized, multiplied and then grown so as to regenerate plantlets. In order to avoid any interference with nontransformed cells, all these operations are carried out on culture media containing the selective agent.

The plants thus regenerated are acclimatized and then grown in a greenhouse, where they may be crossed or self-fertilized.

4.2—Transformation of Maize with Agrobacterium tumefaciens

The transformation of maize with Agrobacterium is carried out according to the method of Ishida et al. (1996).

4.2.1—Production of the Superbinary Vector

The superbinary vector used to transform the maize is derived from homologous recombination between two vectors: the vector PBIOS 371 and the vector pSB1. The vector pBIOS 371, constructed as previously (Example 3.2), comprises, firstly, the T-DNA containing the expression cassette for the OsMIPP gene, inserted between the pHMWG promoter and the Nos terminator, the expression cassette for the selectable gene, the Bar gene. In addition, pBIOS 371 also contains the gene for resistance to spedtinomycin and an origin of replication in E. coli. The vector pSB1 contains the virB, virC and virG genes of the plasmid pTiBo542 present in Agrobacterium strain A281 (ATCC 37349), the gene for resistance to tetracyclin, and an origin of replication which is functional in E. coli and Agrobacterium. The vectors pSB1 and PBIOS 371 possess a homologous region which allows them to recombine and generate the superbinary vector pRec 371.

Homologous recombination between the two vectors takes place in Agrobacterium. The vector PBIOS 371 is introduced into Agrobacterium strain LBA4404 containing the vector pSB1 by electroporation using a CELL PORATOR Voltage Booster device (GIBCO BRL) according to the method described by Mattanovitch et al. (1989) and the protocol given by the supplier (Life Technologies, USA).

The agrobacteria containing the superbinary vector pRec 371 are selected on YT medium in the presence of CaCl₂, rifampicin and spectinomycin. The gene for resistance to rifampicin is carried by the bacterial chromosome.

The resistance to specinomycin, carried by the vector pBIOS 371 (origin of replication functional in E. coli), may only be expressed after homologous recombination with the vector pSB1 (origin of replication functional in Agrobacterium and E. coli).

The superbinary vector pRec 371 possesses the T-DNA which contains the expression cassettes for the Bar gene and for the OsMIPPs sequence (under the control of the PHMWG promoter for tissue-specific expression), origins of replication which are functional both in E. coli and Agrobacterium, the genes for resistance to tetracyclin and to spectinomycin, and the virB, virC and virG virulence genes of the plasmid pTiBo542.

4.2.2—Method of Transformation of Maize and Regeneration of the Transformed Plants

Immature embryos are cocultured with A. tumefaciens strain LBA 4404, containing the superbinary vector, for 5 minutes, and then placed on a callogenesis initiation medium for 5 days in the dark and at 25° C.

The calluses transformed are selected on a culture medium containing the selective agent and the bacteriostatic agent cefotaxim. Type I calluses are obtained and, from these, plantlets will be regenerated on a culture medium containing selective agent and the bacteriostatic agent cefotaxim. The plantlets which have been regenerated are then transferred onto a development medium containing the selective agent.

The plants obtained are acclimatized in a phytotron, and then grown in a greenhouse where they may be crossed or self-fertilized.

4.3—The Bar Gene

The nature and the concentration of the selective agent may vary depending on the gene used. Selective agents which can be used are generally active compounds of certain herbicides (Basta®, Round up®) or certain antibiotics (hygromycin, kanamycin).

The Streptomyces hygroscopicus Bar gene encodes a phosphinothricin acetyltransferase (PAT) which inactivates phosphinotricin—active molecule of the herbicide Basta®—by acetylation. The cells carrying this gene are therefore made resistant to this herbicide and can be selected by it.

For the transformation of cereals, the coding sequence of the Bar gene is under the control of a regulatory region which allows strong and constitutive expression in the plant cells. Such a region may advantageously consist of the promoter and the first intron of the rice actin 1 gene (Mc Elroy et al., 1991).

4.3.1—Use of the Bar Gene in Transformation by Particle Bombardment

The chimeric gene, composed of the promoter and the first intron of the rice Actin I gene and of the Bar gene, is cloned into the plasmid pDM 302 which allows it to be multiplied in E. coli (Cao et al., 1992). The culture media intended for the selection of transformed cells are supplemented with 2 mg/l phosphinothricine.

A technique termed cotransformation may advantageously be used to introduce the OsMIPP constructs which should lead to ectopic expression of the proteins derived from the OsMIPP genes. The two plasmids (the plasmid carrying the Bar gene and the plasmid carrying the OsMIPP gene) are coprecipitated onto the particles of tungsten, the total amount of DNA precipitated on the particles remaining identical to the amount in the standard protocol (5 μg of DNA per 2.5 mg of particles), each plasmid will represent approximately half the total DNA used.

The experiment shows that, with this method, cointegration with the plasmids into the plant cells is the most frequent event (of the order of 90%), i.e. virtually each plant which has integrated the Bar gene and which has been selected by this gene will also carry the MIPP gene. Thus, the percentage of plants selected which express the OsMIPP gene is approximately 70%.

The genes thus introduced are generally released in the genetic sense; the OsMIPP gene can thus advantageously be followed in the descendants by virtue of the herbicide resistance which is closely associated with it.

4.3.2—Use of the Bar Gene in Transformation by Particle Bombardment

The superbinary vector pRec 371 possesses the T-DNA which contains the expression cassettes for the Bar and OsMIPP genes, origins of replication which are functional both in E. coli and Agrobacterium, the genes for resistance to tetracycline and to spectinomycin, and the virB, virC and virG virulence genes of the plasmid pTiBo542.

Immature embryos are cocultured with A. tumefaciens strain LBA 4404, containing the superbinary vector pRec 371, for 5 minutes, and then placed on a callogenesis initiation medium for 5 days in the dark and at 25° C.

Selection of the transformed type I calluses and regeneration of the plantlets are carried out on a medium containing 5 to 10 mg/l phosphinotricine and a bacteriostatic agent, cefotaxim. Development of the plantlets takes place on a medium containing only phosphinotricine.

The calluses and the plants which have integrated into their genome the T-DNA which contains the OsMIPP gene and the Bar gene can be followed in the descendants by virtue of the herbicide resistance.

4.4—Measuring the Phytase Activity of the Transformed Maize

Since the final aim of the production of maize transformed with the OsMIPP gene is to obtain increased phytase activity, measurements of enzymatic activity are planned. Phytase activity is assayed according to the method described previously in Example 2, paragraph 2.5. The phytase activity is measured on the protein extracts of various organs of transgenic maize and of nontransgenic maize: leaves, seeds, young plantlets undergoing germination (5 or 6 days of germination). Extraction of total proteins from these tissues is carried out according to the method described below. The tissues, removed and frozen at −80° C., are ground into powder in liquid nitrogen. A 100 mg amount of ground plant powder is transferred into 1 ml of extraction buffer (100 mM sodium acetate, pH 4.8, 2 mM CaCl₂, 1 mM DTT, 0.5 to 1 mM protease inhibitor cocktail (Roche)). The samples are homogenized for 1 hour at 4° C. and the extracts are centrifuged at 8000 rpm for 20 min at 4° C. The supernatant containing the total proteins is used to measure the phytase activity.

### Example 5

### Production of Transformed Rice

The production of transformed rice can be envisioned according to two methods: via Agrobacterium tumefaciens according to the protocol described by Hiei et al. (1994), by particle bombardment according to the protocol of Fauquet et al. (1996). The vectors pBIOS 360 and pBIOS 371 described in Example 3 are used to transform rice via Agrobacterium tumefaciens.

### Example 6

### Production of Transformed Wheat

The transformation of wheat (Triticum aestivum) may be carried out according to the method described in patent EP 0674 715 B1.

6.1—Preparation of the Tissues to be Transformed

The tissues used for the gene transfer were prepared according to the method described in patent EP 0674 715 B1. Type M calluses are used for the bombardment. They can be obtained from immature embryos taken from the immature grains of wheat and placed on an MS medium described by Murashige and Skoog (1962) containing maltose.

6.2—Preparation of the Vectors to be Transferred

The vectors used to transform the wheat are purified on a cesium gradient and then concentrated to 1 mg/ml in TE buffer, pH 8.0 (10 mM Tris-HCl; 1 mM EDTA) (Sambrook et al., 1989).

6.3—Preparation of Gold Particles

1 μm gold particles are coated with DNA to be transferred, according to the method by Daines (1990). The particles are taken up in absolute ethanol, at 60 mg of particles per ml of ethanol. A 35 μl volume of this suspension is transferred into a 1.5 ml microcentrifugation tube, and the particles are recovered by centrifugation at 14 000 g of 3 minutes. They are washed with 1.5 ml of sterile distilled water and recovered by centrifugation at 14 000 g for 3 minutes. The particles are taken up in 25 μl of TE buffer containing 25 μg of the vector carrying the gene of interest and 25 μg of the vector carrying the selectable gene, the dhfr gene encoding the dihydrofolate reductase. The following are added to this preparation, in this order: 220 μl of sterile distilled water, 250 μl of 2.5 M CaCl₂ and. 50 μl of 0.1 M spermidine. The mixture is homogenized by agitation at 4° C. for 15 minutes. The mixture is centrifuged for 5 minutes at 14 000 g, the supernatant is removed and the particles are washed with 200 μl of absolute ethanol. The DNA-loaded gold particles are taken up in 36 pl of absolute ethanol.

6.4—Transformation Step

The sequence of operation to bombard the cells was carried out as described in patent application EP 0 674 715. In the gun chamber, the Petri dish containing the explants to be bombarded is placed on the platform at the center of the opening from where the DNA-coated gold particles, or microprojectiles, are projected. The microprojectiles are placed on a support above the explants. The chamber is closed and sealed, the chamber is placed under vacuum and the helium reservoir is filled with an appropriate amount of gas.

The bombardment is triggered, allowing projection of the microprojectiles onto the explants. The explants are bombarded twice with the microprojectiles. After bombardment, the calluses are kept in the dark for 15 hours and then placed on MS medium for 2 weeks.

6.5—Selection of the Calluses and Regeneration of the Transgenic Plants

The transformed calluses are selected on the MS medium containing the selective agent, methotrexate, for 4 months.

To regenerate plants, the calluses are transferred onto MS medium containing 2,4-D in the dark. When embryogenic structures appear, the calluses are transferred onto an MS medium containing hormones (auxins and gibberellins), in the light, for 2 weeks; this step allows induction of stems. The explants are then transferred onto a hormone-free MS medium in order to allow induction of roots.

The plantlets are acclimatized in a phytotron before being grown in a greenhouse.

### REFERENCES

Anderson et al. T. A. G. (1989); 77:86-91

Armstrong et al. Maize Genetics Cooperation Newsletter (1991), 65:92-93

Armstrong et al. The Maize Handbook (1994), 665-671

Barba et al. Cell Mol. Biol. (1997) 43:609-620

Berlyne et al. Amer. J. Clinical Nutr. (1973), 26:910-911

Bevan et al., Nature (1983), 304:184-187

Bevan et al. Nucleic Acids Research (1984), 11:369-385

Bradford. Anal. Biochem. (1976), 72:248-254

Cao et al. Plant Cell Reports (1992), 11:586-591

Caffrey et al. FEBS Letters (1999), 442:99-104

Callis et al. Genes Dev. (1987), 1:1183

Carrington and Freed. J. Virol. (1990), 64(4):1590-1597

Chi et al. Genomics (1999), 56:324-336

Chilton et al. Nature (1982), 295:432-434

Christensen et al. Plant Mol. Biol. (1992), 18:675-689

Chupeau et al. Biotechnology (1989), 7(5):503-508

Craxton et al. Biochem. J. (1997), 328:75-81

Daines et al. Biolostic Systems Newsletter (1990)1:1-4

Datla et al. Biotechnology Ann. Rev. (1997), 3:269-296

Depicker et al. Mol. Gen. Genet. (1992), 235(2-3): 389-396

Depigny-This et al. Plant Mol. Biol. (1992), 20:267-479

Elroy-Stein et al. PNAS USA (1989), 86:6126-6130

Fauquet et al. Biolistic transformation of rice: now efficient and routine for japonica and indica rices. Proc. Third Int. Rice Genet Symp. Ed. G. S. Khush, 153-165

Franck et al. Cell (1980), 21:285-294

Gallie et al. Molecular Biology of RNA (1989), 237-256

Gaubier et al. Molecular and General Genetics (1993), 238:409-418

Gibson and Ullah. Plants (1990), pp. 77-92, Wiley-Liss, New York.

Graf E. dir. pub. (1986) Phytic acid, chemistry and applications, Pillsburry Co., Pilatus Press; Minneapolis, Minn., USA

Jose-Estanyol et al., Plant Cell (1992) 4:413-423

Herrera-Estrella et al. EMBO J. (1983), 2:987-995

Hiei et al. The Plant Journal (1994), 6:271-282

Horsch and Klee. Proceeding of the National Academy of Sciences USA (1986), 83:4428-4432

Ishida et al. Nature biotechnology (1996), 14:745-750

Jobling et al. Nature (1987), 325:622-625

Johansen et al. Fifth International Congress of Plant Molecular Biology (1997), abstract No. 1268

Kasuga et al. Nature Biotechnology (1999), 17:287-291

Kay et al. Science (1987), 236:4805

Klein I et al. Nature (1987), 327:70-73

Lechelt et al., Mol. Gen. Genest., (1989), 219:225-234

Laemmli, Nature (1970), 227:680-685

Maas et al. Plant Mol. Biol. (1991), 16:199

Macejack et al. Nature (1991), 353:90-94

Mattanovitch et al. Nucleic Acids Research (1989), 17(16):6747

Mc Cabe et al. Biotechnology (1988), 6(8):923-926

Mc Carty et al., Plant Cell, (1989), 1:523-532

Mc Elroy et al. Plant Cell (1990), 2:163-171

Mc Elroy et al. Mol. Gen. Genet. (1991), 231:150-160

Morris et al. Virology (1992), 187:633

Mullis et al. Method in Enzymology (1987), 155-335

Murashige and Skoog. Physiol. Plant (1962); 15:473

Neuhaus et al. Theoretical and Applied Genet. (1987), 75(1): 30-36

O'Dell et al. J. Agr. Food. Chem (1972), 20:718-721

Ohta et al. Plant Cell Physiol. (1990), 31:805

Pointillard, INRA-Production animales [Animal production] (1994), 7:29-39

Roberts et al. The Plant Cell (1989), 1:569-578

Romano et al. J. Cell Sci. (1998), 111(6): 803-813

Sambrook et al. Molecular Cloning: A Laboratory Manual. Cold Spring Harbor Press, Cold Spring Harbor, N.Y., (1989)

Schell and Van Montagu, Mol. Gen. Genet. (1983), 189(3): 390-399

Schocher et al. Biotechnology (1986), 4:1093-1096

Shan et al. Nutr. Metab. (1979), 23:275-285

Shen et al. Plant Molecular Biology (1994), 26:1085-1101

Snowdon et al. Plant Mol. Biol. (1996), 31:689

Southern. Journal of Molecular Biology (1975), 98:503

Vacanneyt et al. Molecular and General Genetics (1990), 220:245-250

Vain et al. Plant Cell Tissue and Organ Culture (1989), 18:143-151

Verdaguer et al. Plant Molecular Biology (1996), 31(6), 1129-1139

Verdaguer et al. Plant Molecular Biology (1998), 37(6), 1055-1067

Verwoerd et al. Nucleic Acids Research (1989), 17(6): 2362

White et al. Nucleic Acids Research (1990), 18:1062

Zambryski et al. EMBO J. (1983), 2(12): 2143-2150

AU-689311

EP 0 507 689

EP 0 633 317

EP 0 674 715 B1

U.S. Pat. No. 5,188,642

U.S. Pat. No. 5,635,618

U.S. Pat. No. 5,670,349

WO 96/38567

WO 98/05785

WO 98/45445

WO 98/49316

1. An isolated nucleic acid encoding a plant MIPP (Multiple Inositol Polyphosphate Phosphatase) with phytase activity.

2. The isolated nucleic acid according to claim 1, which comprises the sequence SEQ ID No. 1, No. 3 or No. 17 or a sequence homologous to one of these sequences.

3. The isolated nucleic acid according to claim 1, which comprises a sequence homologous to the sequence SEQ ID No. 1, No. 3 or No. 17, said homologous sequence being defined as i) a sequence similar to at least 70% of the sequence SEQ ID No. 1, No. 3 or No. 17; or
ii) a sequence which hybridizes with the sequence SEQ ID No. 1, No. 3 or No. 17, or a sequence complementary thereto, under stringent hybridization conditions, or
iii) a sequence encoding a plant MIPP enzyme comprising the amino acid sequence SEQ ID No. 2, No. 4 or No. 18.

4. The isolated nucleic acid according to claim 1 or 2, which comprises the sequence SEQ ID No. 17 or a sequence homologous to the sequence SEQ ID No. 17.

5. An isolated plant MIPP (Multiple Inositol Polyphosphate Phosphatase) enzyme with phytase activity.

6. The isolated plant MIPP enzyme according to claim 5, which comprises the amino acid sequence SEQ ID No. 2, SEQ ID No. 4 or SEQ ID No. 18.

7. The isolated enzyme according to claim 6, which comprises the sequence SEQ ID No. 18.

8. The isolated plant MIPP enzyme according to claim 5, which comprises an amino acid sequence similar to at least 70% of the sequence SEQ ID No. 2, SEQ ID No. 4 or SEQ ID No. 18.

9. An expression cassette containing at least one of the sequences according to claims 1 to 4, placed under the control of at least one regulatory sequence capable of controlling the expression of the plant MIPP protein.

10. The expression cassette according to claim 9, wherein the regulatory sequence is a promoter.

11. The expression cassette according to claim 10, wherein the promoter is specific for an organ or for a tissue of the plant.

12. The expression cassette according to claim 11, wherein the specific promoter is selected from the promoters of genes encoding a wheat or barley high molecular weight glutenin (HMWG), napin, phaseolin, helianthinin, albumin, oleosin, GEAL and GEA6 of Arabidopsis thaliana, maize γ-zein, the pHyPRP promoter of the gene encoding a maize hybrid proline rich protein, and the Vp1 promoter of the Viviparous-1 gene combined with the first intron Sh of the Shrunken gene.

13. The expression cassette according to claims 9 to 12, wherein a regulatory sequence comprises an addressing signal.

14. The expression cassette according to claim 13, wherein the addressing signal is a sequence encoding a N-terminal signal peptide.

15. The expression cassette according to claim 14, wherein the addressing signal comprises an endoplasmic retention signal at the C-terminal end.

16. The expression cassette according to claim 14, wherein the addressing signal does not comprise an endoplasmic retention signal at the C-terminal end.

17. A nucleotide vector into which an expression cassette as defined in one of claims 9 to 16 is inserted.

18. A host cell containing a nucleotide vector according to claim 17.

19. A method for producing transgenic plants comprising the steps of:
transforming plant cells with a nucleic acid sequence according to any one of claims 1 to 4 or a vector according to claim 17,
selecting the transformed cells,
generating the transformed plants from these cells, expressing a plant MIPP with phytase activity encoded by said nucleic acid sequence.

20. A transformed plant which can be obtained using the method of claim 19.

21. A part of a plant, in particular a seed, which originates from a transformed plant according to claim 20.

22. A product, in particular a flour, which can be obtained from a transformed plant according to claim 20 or from a part of a transformed plant according to claim 21.

23. A method for producing a plant MIPP protein, which comprises the steps of:
a) transforming a cell, in particular of a plant or of a microorganism, with an expression cassette according to any one of claims 9 to 16,
b) optionally culturing said host cell or, when this host cell is a plant cell, growing the transformed plant,
c) extracting the MIPP protein from the cell culture or from the transformed plant.

24. The use of a plant or part of a plant according to claim 20 or 21., or else of a product according to claim 22, in any situation in which phytase activity is necessary or desired.

25. The use according to claim 24, for releasing inorganic phosphate and/or improving the availability of the cations chelated by phytin, such as iron, calcium, magnesium or zinc.

26. The use of an expression cassette according to any one of claims 9 to 16, for obtaining a transgenic plant which produces seeds containing MIPP.
